(19) United States
(12) Patent Application Publication
(10) Pub. No.: US 2023/0227487 A1
(43) Pub. Date: Jul. 20, 2023

(54) IMPROVED DEMINERALIZATION OF FERMENTATION BROTHS AND PURIFICATION OF FINE CHEMICALS SUCH AS OLIGOSACCHARIDES

(71) Applicant: BASF SE, Ludwigshafen am Rhein (DE)

(72) Inventors: Johan Fredrik BILLING, Ludwigshafen am Rhein (DE); Emiel Jan KAPPERT, Ludwigshafen am Rhein (DE); Chung Huan WONG, Ludwigshafen am Rhein (DE); Jacek MALISZ, Ludwigshafen am Rhein (DE)

(21) Appl. No.: 18/008,684

(22) PCT Filed: Jun. 10, 2021
(86) PCT No.: PCT/EP2021/065685

§ 371 Date: Dec. 7, 2022

(30) Foreign Application Priority Data
Jun. 12, 2020 (EP) 10179785.9

(51) Int. Cl. C07H 1/08; C07H 3/06; A23C 9/142; A23C 9/146

(52) U.S. Cl. CPC C07H 1/08; A23C 9/142; A23C 9/146; C07H 3/06; C12P 19/04

(57) ABSTRACT

The present invention relates to a method for improved demineralization of fermentation broths, including the steps of providing a solution comprising one or more oligosaccharides, carrying out a first membrane filtration and preferably being a microfiltration or ultrafiltration, a second membrane filtration of the permeate of the first membrane filtration and a first nanofiltration step; with a sub-step of concentration and/or a sub-step of diafiltration. Moreover, the present invention is directed to an improved purification of fine chemicals from a fermentation broth.

### TECHNICAL FIELD

The present invention relates to a method for separating biomass from a solution comprising biomass and at least one oligosaccharide.

### BACKGROUND

Human milk oligosaccharides (HMOs) are the third most abundant solid component of human milk after lactose and lipids. The concentrations of different HMOs and their total amount in human milk vary within the lactation phase and between individuals, which is believed to be partially based on genetic background. Importantly, however, HMOs are not found in comparable abundances in other natural sources, like cow, sheep, or goat milk. Several beneficial effects of HMOs on infants have been shown or suggested, including selective enhancement of bifidobacterial growth, anti-adhesive effects on pathogens and glycome-altering effects on intestinal epithelial cells. The trisaccharide 2′-fucosyllactose (2′-FL) is one of the most abundant oligosaccharides found in human milk. Due to its prebiotic and anti-infective properties, 2′-FL is discussed as nutritional additive for infant formula. Moreover, infants’ nutrition containing 2′-FL is associated with lower rates of diarrhea, making 2′-FL a potential nutritional supplement and therapeutic agent, if it were available in sufficient amounts and at a reasonable price.

Formerly, 2′-FL has been obtained via extraction from human milk or chemical synthesis, but the limited availability of human milk or the necessity of side group protection and deprotection in chemical synthesis, respectively, set limits to supply and cost efficiency. Thus, alternative sources of 2′-FL became of interest. Besides chemical synthesis and extraction from human milk, 2′-FL can be produced enzymatically in vitro and in vivo. The most promising approach for a large-scale formation of 2′-FL is the whole cell biosynthesis in Escherichia coli by intracellular synthesis of GDP-L-fucose and subsequent fucosylation of lactose with an appropriate α1,2-fucosyltransferase.

Thus, HMOs may be produced by means of fermentation providing a solution comprising biomass and at least one oligosaccharide, preferably 2′-FL. Such a solution may also be called fermentation broth.

Biomass separation from the fermentation broth from the HMO process is the first downstream processing step in the production of HMO. The state-of-the-art technology for this step is centrifugation and or filter press, sometimes with the use of flocculants. However, microfiltration can also be employed and has several advantages in comparison to other separation technologies. To enable a genetically modified organism free product solution, microfiltration is the best option because it can completely retain all non-dissolved solids including genetically modified cells such as microorganisms.

Nanofiltration has been described as a method in the purification for separating HMO tri-and oligosaccharides from lactose (see international patent application published as WO 2019/003133)

### SUMMARY

The invention discloses a novel method for the processing of fermentation broths comprising one or more fine chemical, for example oligosaccharide and biomass up to a purified fine chemical solution or solid fine chemical. The methods employ a particular sequence of steps before the biomass is removed, the removal of biomass and then the demineralization of the solution comprising the desired fine chemical(s). Further the invention is also a method for demineralisation of solutions comprising one or more oligosaccharides by nanofiltration and a rapid purification method for such solutions.

Membrane filtrations are often used to separate smaller molecules from larger ones in a solution. One example for oligosaccharide containing solutions is disclosed in the Chinese patent application published as CN 100 549 019 and CN 101 003 823, a patent application disclosing a method for preparing high-purity xylooligosaccharide from straw by using enzyme and membrane technology. The international application published as WO 2017/205705 discloses the use of membrane filtration for hemicellulose hydrolysis solutions. Another example is disclosed in EP 2 896 628, a patent application disclosing a membrane filtration of oligosaccharide containing fermentation broth followed by performing further process steps including addition of activated carbon to the filtrate.

The separation of the biomass after fermentative production of HMO is usually done at a pH value of 7 by means of an initial centrifugation or filter press and further centrifugations. Sometimes polymeric membranes are used instead.

When membranes are used, however, the membrane performance is rather low and the permeate contains a high amount of proteins and colour components, which have to be removed in the following steps leading to an elaborate downstream process, high product yield losses and some quality problems.

Typically, after these initial steps of biomass separation from fermentation broths the next step carried out is an ultrafiltration completed typically with 10 kDa polyethersulfone membranes, yet not all proteins and polysaccharides can be separated by this. The ultrafiltration permeate is hence sent to an active carbon column to decolourize the solution and achieve an APHA value of below 1000. The decolourization in the active carbon column is a rather tedious process and it is often necessary to use around 14% weight/weight of active carbon in relation to the initial amount of fermentation broth. This step leads to high product losses and necessitates huge active carbon columns.

After ultrafiltration and decolourization, the solution comprising a fine chemical such as an HMO is often subjected to an ion exchange treatment to reduce the charged side components. Large volumes of solution may require large ion exchange equipment and large amounts of ion exchange resins. A concentrations step may be used to reduce volumes before the ion exchange hence.

The international application published as WO2015106943 discloses a procedure in which an ion exchange step may follow a nanofiltration step after initial membrane filtrations. However, repeated decolourization is required and the ion exchange step is not a removal of salts. Cations are removed to be replaced by NaOH, and Anions are exchanged for Chloride ions. The removal of the salts then is achieved by an additional electrodialysis step.

The invention concerns a method that advantageously combines a decolourization of the solution comprising the fine chemical e.g. HMO after fermentation, with a biomass separation process and with a specific sequence of purification steps of nanofiltration before demineralization for example but not limited to ion exchange, or nanofiltrations without any subsequent demineralization e.g. ion exchange step(s).

The first part of the inventive method allows for a biomass separation and decolourization that is lean on resources and allows for the second part of the method, an advantageous purification of the desired fine chemical without any further preparations or additions of ions.

In the inventive method nanofiltration is used to remove salts, preferably monovalent ions and low molecular weight side components in addition to concentration of the products. By doing so, the ion exchanger can operate at higher concentrations of product and needs to remove less side components, which allows the size of the operation to be decreased strongly. Moreover, the surprising effectiveness of the overall purification process including nanofiltration allows for a replacement of an ion exchange step by a nanofiltration.

In a preferred embodiment of the present invention, the method for purification of one or more fine chemicals, preferably oligosaccharides and or aroma compounds, from a solution comprising biomass and one or more fine chemical is a method for comprising the steps of:
i. providing a solution comprising biomass and one or more fine chemical,
ii. setting the pH value of the solution below 7, preferably below pH 5.5 or less by adding at least one acid to the solution comprising biomass and the at least one oligosaccharide,
iii. adding an adsorbing agent to the solution comprising biomass and fine chemical,
iv. Optionally an incubation step,
v. carrying out a membrane filtration also called herein the first membrane filtration and typically being a microfiltration or ultrafiltration so as to separate the biomass from the solution comprising the at least one fine chemical;
vi. Optionally carrying out at least one second or further membrane filtration with the permeate of the first membrane filtration, preferably at least one ultrafiltration;
vii. Optionally carrying out a decolourization step
viii. Carrying out a nanofiltration (step S22, see FIG. 1) with the permeate of the membrane filtration antedating this step S22, either with the permeate of the first or the second or any further membrane filtration;
ix. Optionally a decolourization step;
x. Optionally a second nanofiltration S24 with the retentate of the nanofiltration of the previous nanofiltration step i, wherein the nanofiltration membrane used is a different one to the one in the previous nanofiltration step S22;
xi. Optionally a third or further nanofiltration with a membrane differing from the one of the previous nanofiltration step;
xii. Further processing of the retentate of the previous nanofiltration step by any of the following steps:
   a. Optionally carrying out a decolourization step.
   b. Optionally carrying out a demineralization step, more preferably a cation exchange and / or anion ion exchange, or
   c. Optionally carrying out an electrodialysis and / or reverse osmosis and / or concentration step and / or a decolourization step
   d. Optionally carrying out a simulated moving bed chromatography, and / or a solidification step creating a solid fine chemical product, preferably a crystallisation step and / or a spray drying of the fine chemical followed by drying as desired.

For the easier storage and transport, it is often desirable to have the fine chemical such as an oligosaccharide in solid form rather than in solution. Hence, in a preferred embodiment the inventive method as a final step has the removal of the desired fine chemical(s) for example oligosaccharide(s) like one or more HMO from the solution. This may be done by crystallisation for example but not limited to crystallisation with the help of one or more solvents such as but not limited to short chain alcohols (e.g. methanol, ethanol, propanol, butanol) and / or organic acids, preferably food-grade organic acids such as but not limited to acetic acid and/or propionic acid. Alternatively, removal from the solution may be achieved in said final step of the inventive method by spray-drying or any other method for removal of water or solvent from the desired fine chemical to a suitable dryness of the fine chemical. Also, such steps of removing the fine chemical from the solution may be employed before said final step. For example the inventive method encompasses steps of crystallisation or spray drying followed by re-dissolving the fine chemical to create a new solution, optional other purification steps or repetitions of the removal from solution and re-dissolving of the fine chemical to form a new solution and then as a final step removal from the solution again.

The nanofiltration of step S22 may include sub-steps of concentration and diafiltration, also called washing, in alternation, starting in any order. However, when first a diafiltration mode is used, large volumes of amounts of a diafiltration medium, typically deionized water, are required. Therefore, in a preferred embodiment, a nanofiltration step S22 with sub-steps in which first concentration sub-step, then diafiltration sub-step and then optionally again concentration sub-step is performed.

In a preferred embodiment, after the nanofiltration S22 or after the second nanofiltration, there is no addition of anions and /or cations, e.g. NaOH, in any subsequent steps except short chain organic acids for crystallisation. In particular, the inventive method allows to remove salts without the use of electrodialysis and in a preferred embodiment without the use of ion exchange to arrive at a solid product.

According to the method of the present invention, it was surprisingly found, that the membrane performance in the first membrane filtration can be significantly increased, and removal of proteins can be significantly improved when the pH value of the solution is lowered to below 7 before carrying out the first membrane filtration. Further, it was found that membrane performance increases further and the colour of the permeate can be significantly reduced to values below the required specification when an adsorbing agent is added to the solution before any membrane filtration. Also advantageously, the needed amount of adsorbing agent like active carbon is much lower as compared to the known methods, and also the required time for decolourization is much shorter than in known methods, when the membrane filtration is done after the pH value has been set to the desired target value below pH 7 and at least one adsorbing agent has been added.

Preferably, the adsorbing agent is active carbon. Active carbon, also known as activated carbon or activated charcoal, is a preferred adsorbing agent as it is of low cost, available in large quantities, easy to handle and safe for use in conjunction with foodstuffs.

It is beneficial to the methods of the invention that the pH value of the solution comprising biomass and one or more oligosaccharide, one or more disaccharide and / or one or more monosaccharide is below pH 7.0 when the first membrane filtration is performed, and more preferably when the adsorbing agent is added. Hence, since pH values of fermentation broth are typically at or above pH 7.0, the pH value is lowered by the addition of at least one acid as needed to achieve the target pH value. In case the pH value of the solution comprising biomass and one or more oligosaccharide, one or more disaccharide and / or one or more monosaccharide is already below pH 7.0 at the start, at least one acid may be used for setting the pH value stably below pH7.0 as needed. Also, preferably, the pH value of the solution is set to a pH value of 5.5 or below, before any membrane filtration is started. Preferably the pH value is lowered to a target pH value in the range of 3.0 to 5.5, more preferably the range of 3.5 to 5, wherein the ranges given include the given numbers. In an even more preferred embodiment, the pH value of the solution is set to pH 3.5 or above, but not higher than pH 4.5 and most preferably the pH value is set to a value in the range of and including 4.0 to 4.5. To this end, at least one acid is added to the solution. Said at least one acid is, more preferably, an acid selected from the group consisting of H₂SO₄, H₃PO₄, HCl, HNO₃ and CH₃CO₂H. Basically, any acid may be used. Nevertheless, these acids are usually easy to handle.

Said adsorbing agent, preferably active carbon, is typically added in an amount in the range of 0.25% to 3% by weight, preferably in the range of 0.5% to 2.5% by weight and more preferably in the range of 0.75% by weight to 2.2% by weight and even more preferably in the range of 1.0% to 2.0% by weight, wherein the percentage values are on a weight of adsorbing agent per weight of solution basis. Thus, a rather small amount of said adsorbing agent, preferably active carbon, is sufficient to reduce the colour number below the upper bound specification, which is preferably 1000 APHA. This allows for significant reduction of active carbon consumption as well as for significant reduction of product losses in comparison to the active carbon column. In one embodiment one or more adsorbing agents are added in an amount suitable to bind - in increasing order of preference - at least 50%, 55%, 60%, 65%, 70%, 75%, 80%, 90%, 92%, 94%, 95% or more of the colour components and / or the protein in the starting solution comprising biomass and / or polysaccharides and / or proteins and / or nucleic acids like DNA or RNA that may be present. Further, said adsorbing agent, preferably active carbon, is typically added as a powder having a particle size distribution with a diameter d50 in the range of 2 µm to 25 µm, preferably in the range of 3 µm to 20 µm, for example those with a d50 of 10 to 15 µm, and more preferably in the range of 3 µm to 7 µm, and even more preferably in the range of 5 µm to 7 µm. Such powder of low d50 values can be made by wet milling of larger powders. The d50 value is determined with standard procedures. Particle sizes in this size range reduce the risk of abrasion of the membrane. Moreover, said adsorbing agent, preferably active carbon, is yet preferably added as a suspension of the powder in water. This facilitates handling of the adsorbing agent as the suspension of the powder may better mix with the suspension comprising biomass and the oligosaccharide. The adding of said adsorbing agent, preferably active carbon, to the solution is, typically, carried out after adding the at least one acid to the solution. Unexpectedly, the colour reduction and protein reduction are much better, when the pH value is adjusted first and then the adsorbing agent or at least the majority of the adsorbing agent is added subsequently. It is possible to add said adsorbing agent, preferably active carbon, to the fermentation broth before adding the at least one acid to the solution.

In another variant, the pH value of the solution is lowered to 5.5, more preferably to 5.0 and even more preferably to 4.5 by the addition of at least one of the suitable acids, and then adsorbing agent, preferable active carbon, and further acid is added until the desired final pH value is achieved.

Also, some of the adsorbing agent may be added before any acid is added to lower the pH value, followed by the addition of more adsorbing agent after the pH value has been set to the target value below pH 7.0.

Preferably, said solution comprising biomass and at least one fine chemical, preferably an oligosaccharide or aroma compound, typically is a fermentation broth, obtained by cultivation of one or more types of cells, preferably bacteria or yeast, more preferably bacteria, even more preferably genetically modified Escherichia coli, Amycolatopsis sp. or Rhodobacter sphaeroides., in a cultivation medium, preferably a cultivation medium comprising at least one carbon source, at least one nitrogen source and inorganic nutrients. Thus, sufficient amounts of said fine chemical(s), preferably oligosaccharide(s), may be produced with cost efficient methods.

Said microfiltration or ultrafiltration of the first membrane filtration step is typically carried out as cross-flow microfiltration or cross-flow ultrafiltration. Thus, the filtration efficiency may be enhanced. Said cross-flow microfiltration or cross-flow ultrafiltration includes a cross-flow speed above 0.2 m/s, preferably in the range of 0.5 m/s to 6.0 m/s, more preferably in the range of 2.0 m/s to 5.5 m/s and even more preferably in the range of 2.8 m/s to 4.5 m/s, and most preferably in the range of 3.0 m/s to 4.0 m/s if ceramic mono- and multi-channel elements are used. In another embodiment, the cross-flow speed is equal to or below 3.0 m/s. In case that a polymeric membrane is used for the first membrane filtration, cross-flow speeds of 2 m/s or less can be used; cross-flow speeds in the range of 0.5 m/s to 1.7 m/s are preferably used, but even cross-flow speeds of 0.5 m/s or less may be used. In another preferred embodiment, the cross-flow speed is not more than 1.7 m/s, 1.6 m/s, 1.5 m/s, 1.4 m/s, 1.3 m/s, 1.2 m/s, 1.1 m/s or 1.0 m/s if a polymeric membrane is used. Thus, the filtration speed may be optimized when compared to a filtration process without including a pH value adjustment and addition of an adsorbing agent. By doing so, wear and tear on and/or energy consumption of the membrane filtration equipment can be reduced by operating at lower cross-flow speed compared to previously known methods, while resulting in good separation.

Said first membrane filtration, preferably a microfiltration or ultrafiltration is, typically, carried out at a temperature of the solution in the range of 4° C. to 55° C., preferably in the range of 10° C. to 50° C. and more preferably in the range of 30° C. to 40° C. Thus, the temperature during said filtration step may be the same as during fermentation which further improves the membrane performance and decreases viscosity of the solution comprising biomass and oligosaccharide. Yet, the first membrane filtration is, also preferably, carried out by means of a ceramic microfiltration membrane or ceramic ultrafiltration membrane having a pore size in the range of 20 nm to 800 nm, preferably in the range of 40 nm to 500 nm and more preferably in the range of 50 nm to 200 nm. It is also possible to use multi-layered membranes that are engineered to have improved abrasion resistance, e.g. 400 nm and 200 nm and 50 nm pore size layers of Al₂O₃.Thus, sufficient amounts of proteins and polysaccharides may be removed in order to comply with the desired specification. Also typically, first membrane filtration is carried out by means of a polymeric ultrafiltration membrane having a cut-off above or equal to 4 kDa, preferably above or equal to 10 kDa, more preferably equal to or above 50 kDa and even more preferably equal to or above 100 kDa. Also typically, first membrane filtration is carried out by means of a polymeric microfiltration membrane having a pore size of 200 nm or less, preferably of 100 nm or less. Thus, sufficient amounts of proteins and polysaccharides may be removed in order to comply with the desired specification.

The polymeric material of the polymeric microfiltration membrane or polymeric ultrafiltration membrane is, preferably, at least one polymeric material selected from the group consisting of: polyethersulfone, polysulfone, polypropylene, polyvinylidene fluoride, polyacrylonitrile, polyvinylidene fluoride. Modified polymeric materials can also be used, for example hydrophilized polyethersulfone.

The ceramic material of the ceramic microfiltration membrane or ceramic ultrafiltration membrane is, preferably, at least one ceramic material selected from the group consisting of: TiO2, ZrO₂, SiC and Al₂O₃.

The first membrane filtration, preferably microfiltration or ultrafiltration is, typically, carried out after a predetermined time after the adsorbing agent, preferably active carbon, has been added to the solution. This allows to provide an adsorption time during which colour components are adsorbed. Said predetermined time is at least 2 min, preferably at least 10 min and more preferably at least 20 min. Thus, the adsorption of colour components is rather quick.

The method may, preferably, further comprise carrying out a second or further membrane filtration, preferably an ultrafiltration, using the solution essentially free of biomass obtained by the microfiltration or ultrafiltration of the first membrane filtration and comprising one or more oligosaccharide, one or more disaccharides and / or one or more monosaccharides, preferably comprising the majority of these saccharides from the starting solution, e.g. the fermentation broth, that also comprised the biomass . Preferably, the second membrane filtration is done with the permeate of the first membrane filtration and with a membrane having a lower cut-off than the first membrane. Thus, an advantageous further processing of the permeate obtained by the first membrane filtration is realized. The second membrane filtration is, typically, an ultrafiltration carried out by means of an ultrafiltration membrane, preferably, at least partially made of a polymeric material, and having a cut-off in the range of 1 kDa to 10 kDa, preferably in the range of 2 kDa to 10 kDa and more preferably in the range of 4 kDa to 5 kDa. Polymeric membranes typically offer the advantage over tight ceramic membranes that they are more robust and less expensive.

The second membrane filtration may be performed with a ceramic membrane of 1 to 25 kDa, preferably 2 to 10 kDa, more preferably 2 to 5 kDa cut-off. In a further embodiment it is preferable that the membrane is at least partially made of a polymeric material. Said polymeric material is, more preferably, at least one polymeric material selected from the group consisting of: polyethersulfone, polysulfone, polyacrylonitrile, cellulose acetate. Said second membrane filtration is, typically, carried out after adjusting the temperature of the solution to temperatures of below 20, preferably at a temperature of the solution being in the range of 4° C. to 15° C., preferably in the range 8° C. to 13° C. and more preferably in the range 8° C. to 12° C.

In a preferred embodiment, the first membrane filtration employed in the inventive methods includes two or preferably three steps as will be explained in further detail below. The first step includes a first diafiltration having a diafiltration factor DF (amount of diafiltration water = starting amount of fermentation broth × diafiltration factor) ranging from 0.5 or less to 3 or above. For example, for 2′FL comprising solutions it was advantageous to have a DF of 0.5 while for other HMO molecules values of 3 proved to be better if a concentration step was to follow. During diafiltration, the amount of water or a suitable aqueous solution added is identical to the amount of permeate discharged. In a batch wise diafiltration, the volume in the feed vessel is thus kept constant. The second step includes concentrating of the fermentation broth preferably with a factor 2 or more by stopping the feed of diafiltration water and the level will decrease down to the target value (target value = volume or mass at the beginning of the fermentation broth / concentrating factor). Optionally, the subsequent third step includes a second diafiltration. By means of these three steps a lower dilution of the product within the permeate and an increased yield of ≥ 95% are realized. By increasing the factor of the second diafiltration, the yield may even be further increased. However, the dilution of the product will also increase.

The permeate then typically is the combination of all solutions passing through the membrane in these three steps. In a batch process each step produces a permeate fraction in a time-separated manner, that can be collected in one vessel for mixing, or processed separately. In a continuing process, each of the three steps produces a permeate fraction not in a time separated, and these fractions can be combined to form the permeate combined or treated separately if desired.

Optionally the first step of the first membrane filtration may be repeated one or more times, before the second step of concentration is done. Optionally, the second step may be performed, or it may be skipped if concentrating the solution is not desirable. This is useful when the fermentation broth has a high viscosity and or very high biomass content, for example.

Optionally the first step may be skipped and alternatively the second step is done without the first step, so that first a concentration of the fermentation broth is done while creating permeate, and then a diafiltration of the last step is done by feeding water or aqueous solutions to the solution comprising biomass and one or more oligosaccharide, disaccharide or monosaccharide.

Preferably, the at least one oligosaccharide comprises human milk oligosaccharide, preferably neutral or sialylated human milk oligosaccharide and more preferably Lacto-N-tetraose, Lacto-N-neotetraose, 3′-sialyllactose, 6′-sialyllactose and/or 2′-fucosyllactose, and even more preferably 2′-fucosyllactose, 6′-sialyllactose and/or Lacto-N-tetraose.

In one embodiment of the invention, the methods of the invention are applied for the separation of mono-and/or disaccharides from biomass from a solution containing mono-and/or disaccharides and biomass, for example for the separation of lactose, fucose, maltose or saccharose from biomass and the subsequent purification of the mono- and / or disaccharides.

A further embodiment is the inventive apparatus suitable to perform the methods of the invention.

Further features and embodiments of the invention will be disclosed in more detail in the subsequent description, particularly in conjunction with the dependent claims. Therein the respective features may be realized in an isolated fashion as well as in any arbitrary feasible combination, as a skilled person will realize. The embodiments are schematically depicted in the figures. Therein, identical reference numbers in these figures refer to identical elements or functionally identical elements.

### DETAILED DESCRIPTION

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once.

Further, as used in the following, the terms “particularly”, “more particularly”, “specifically”, “more specifically”, “typically”, “more typically”, “preferably”, “more preferably” or similar terms are used in conjunction with additional / alternative features, without restricting alternative possibilities. Thus, features introduced by these terms are additional / alternative features and are not intended to restrict the scope of the claims in any way. The invention may, as the skilled person will recognize, be performed by using alternative features. Similarly, features introduced by “in an embodiment of the invention” or similar expressions are intended to be additional / alternative features, without any restriction regarding alternative embodiments of the invention, without any restrictions regarding the scope of the invention and without any restriction regarding the possibility of combining the features introduced in such way with other additional / alternative or non-additional / alternative features of the invention.

As used herein, the term “biomass” refers to the mass of biological material comprised in the solution of the one or more fine chemical. Typically, said biological material in accordance with the present invention are one or more types of prokaryotic or eukaryotic organisms, or parts thereof of high molecular mass, such as cell walls, proteins, phospholipids, cell membranes, polynucleotides and other large organic compounds produced by the organism.

The biomass may be in suspension and / or also in solution.

Preferably biomass is to be understood to be non-complex biomass, typically of low or no organisation of the cells into peculiar or complex structures. Non-limiting examples are individual cells, cell pairs, cell lumps, oligocellular or multicellular structures, cell layers, biofilms. Non-complex biomass may be of a three-dimensional structure due to a matrix provided by human intervention to the non-complex biomass, for example — but not limited to — cells in a petri dish forming a layer or forming a biofilm lining in a biological reactor, cells or proteins attached to a surface or in a biosensor made by man and the like. The non-complex biomass may be in a three-dimensional structure that is non-natural, but sometimes mimicking a naturally occurring structure, but is only achieved by human intervention.

In contrast to this, complex biomass is to be understood to be of a complex structure by nature, often a complex three-dimensional structure and comprise many hundreds, thousands, ten-thousands, but more typically hundreds of thousands or millions or more of cellular structures in a complex organisation, often of various cell types with different specialisations. Non-limiting examples are higher plants or animals with a body visible with the naked eye, organs and tissues, including bone and meat or plant parts like fruit, vegetables, straw, sugarcane bagasse, hay, wood, timber. Complex biomass may be the source of non-complex biomass, for example cell lines are typically derived from a tissue or organ but do not maintain the complex structure in cultivation.

In a more preferred embodiment, biomass refers to one or more cells of one or more biological organisms, more preferably the one or more organism is a bacterium or a fungal (including yeast) organism or a plant or non-human animal, -and their cellular parts such as but not limited to cell walls, cell organelles, proteins, phospholipids, cell membranes, polynucleotides or polysaccharides. In a more preferred embodiment, the one or more organism is a cell selected from a) the group of Gram negative bacteria, such as Rhodobacter, Agrobacterium, Paracoccus, or Escherichia; b) a bacterial cell selected from the group of Gram positive bacteria, such as Bacillus, Corynebacterium, Brevibacterium, Amycolatopis; c) a fungal cell selected from the group of Aspergillus (for example Aspergillus niger), Blakeslea, Peniciliium, Phaffia (Xanthophyllomyces), Pichia, Saccharamoyces, Kluyveromyces, Yarrowia, and Hansenula; or d) a transgenic plant or culture comprising transgenic plant cells, wherein the ocell is of a transgenic plant selected from Nicotiana spp, Cichorum intybus, Iacuca sativa, Mentha spp, Artemisia annua, tuber forming plants, oil crops and trees; e) or a transgenic mushroom or culture comprising transgenic mushroom cells, wherein the microorganism is selected from Schizophyllum, Agaricus and Pleurotisi. More preferred organisms are microorganism belonging to the genus Escherichia, Saccharomyces, Pichia, Amycolatopsis, Rhodobacter, and even more preferred those of the species E.coli, S.cerevisae, Rhodobacter sphaeroides or Amycolatopis sp. for example but not limited to Amycolatopsis mediterranei, for example the strain NCIM 5008,Streptomyces setonii, Streptomyces psammoticus, and for example but not limited to Amycolatopsis sp strains IMI390106, Zyl 926, ATCC39116, DSM 9991, 9992 or Zhp06.

More preferably, the said biomass comprises organisms or cells thereof and their cellular parts, even more preferably genetically modified organisms or cells, which are cultivated in a cultivation medium, preferably a cultivation medium comprising at least one carbon source, at least one nitrogen source and inorganic nutrients.

The easiest way to assess the success of separating the biomass and fine chemical, preferably the oligosaccharide(s), disaccharide(s) and/ or monosaccharide(s) is to monitor that the permeate of the first membrane filtration is optically clear. Unsuccessful separation will result in biomass being detected in the optical check of the permeate, and the presence of adsorbing agent like black active carbon in the permeate will also easily be detected in the optical check and indicate a leak or failure of the membrane filtration equipment.

As used herein, the term “fine chemical” refers to an oligosaccharide, disaccharide, monosaccharide, aroma compound, polymer, monomer, vitamin, amino acid, peptide, glucoside, nucleic acid, nucleotide. In a preferred embodiment, fine chemical refers to an oligosaccharide or disaccharide or monosaccharide, more preferably to an oligosaccharide, and even more preferably to a human milk oligosaccharide.

As used herein, the term “oligosaccharide” refers to a saccharide polymer containing a small number of typically three to ten of monosaccharides (simple sugars). Preferably, said oligosaccharide comprises human milk oligosaccharide, preferably neutral, acidic nonfucosylated and/or acidic fucosylated, more preferably 2′-fucosyllactose, Difucosyllactose, Lacto-N-tetraose, Lacto-N-neotetraose, LNFP I, LNFP II, LNFP III, LNFP V, LNDFH I, LNDFH II and/or sialic acid containing human milk oligosaccharides such as but not limited to 3′-sialyllactose and/or 6′-sialyllactose, even more preferably 2′-fucosyllactose.

As used herein, the term “disaccharide” refers to a saccharide consisting of two monosaccharides, for example lactose that consists of a glucose and a galactose moiety, or saccharose that is made from one glucose and one fructose molecule.

As used herein, the term “monosaccharide” refers to a simple sugar, preferably a sugar molecule comprising 5 or 6 carbon atoms, for example glucose, fructose, galactose or fucose.

As used herein, the term “aroma compound” refers to any substance that is an odorant, aroma, fragrance, or flavour, and preferably is a chemical compound that has a smell or odour. Preferably an aroma compound is an organic compound typically with a molecular mass up to 1000 Da, preferably up to 800 Da, more preferably up to 600 Da, even more preferably up to 400 Da as a molecular weight. Preferably the aroma compound is a polar aroma compound, even more preferably is selected from the list of furaneol, benzoic acid, phenylethanol, raspberry ketone, pyrazines, vanillin, vanillyl alcohol and vanilla glycoside, and yet even more preferably it is selected from vanillin, vanillyl alcohol and vanilla glycoside.

The term “adsorbing agent” as used herein refers to an element configured to provide the adhesion of atoms, ions or molecules from a gas, liquid or dissolved solid to a surface. The term “adhesion” refers to the tendency of dissimilar particles or surfaces to cling to one another. Preferably, the adsorbing agent is configured to provide adhesion for colour components. Preferably, the adsorbing is active carbon.

As used herein, the term “microfiltration” refers to a type of physical filtration process where a fluid comprising undesired particles, for example contaminated fluid, is passed through a special pore-sized membrane to separate cells such as microorganisms and suspended particles from process liquid, particularly larger bacteria, yeast, and any solid particles. Microfiltration membranes have a pore size of 0.1 µm to 10 µm. Thereby, such membranes have a cut-off for a molecular mass of more than 250 kDa.

As used herein, the term “ultrafiltration” refers to a type of physical filtration process where a fluid comprising undesired particles, for example contaminated fluid, is passed through a special pore-sized membrane to separate cells such as microorganisms and suspended particles from process liquid, particularly bacteria, macromolecules, proteins, larger viruses. Ultrafiltration membranes have typically a pore size of 2 nm to 100 nm and have a cut-off for a molecular mass of 2 kDa to 250 kDa. The principles underlying ultrafiltration are not fundamentally different from those underlying microfiltration. Both of these methods separate based on size exclusion or particle retention but differ in their separation ability depending on the size of the particles.

As used herein, the term “nanofiltration” refers to a type of physical filtration process where a fluid comprising undesired particles, for example contaminated fluid, is passed through a special pore-sized membrane to separate larger compounds from smaller compounds in the solution. It uses membranes with smaller pores than ultrafiltration membranes. An example of nanofiltration membranes are those having pore sizes from 1-10 nm. Nanofiltration membranes are characterized by their at least partial but not complete retention of inorganic salts, such as NaCl or MgSO₄. Because of their retention of these lower molecular species, they are typically operated at higher pressures than ultrafiltration membranes. Herein, “nanofiltration” is defined as a filtration conducted with a membrane having a retention for NaCl between 5 to 90 %, wherein the retention is determined at a salt concentration of 2,000 ppm, a temperature of 25° C., a feed pressure of 8 bar and a recovery of 15 %.

It is understood in the art that the pore size of a filtration membrane is not the only determinant if a membrane filtration is considered a microfiltration, ultrafiltration, nanofiltration or a reverse osmosis. The skilled artisan will be able to distinguish between these methods.

As used herein, the term “solidification” refers to a process of transferring a compound, for example a fine chemical, from a non-solid state of matter to solid state. The compound may be present as a suspension or as a non-suspended solid. Non-limiting examples are crystallisation, spray drying, boiling to dryness, precipitation and flocculation. The solid particles may be dried partly or completely after or as part of the solidification process and still contain solvents like water, or even be suspended in such solvents. For some applications suspensions or slurries are preferred, for other more or less dry solids for examples as powders or crystals are preferred. For compounds not in a solid state at room temperature or to support the solidification process for those that are in a solid state at room temperature, the solidification may be performed at a lower temperature. Non-limiting examples are cooling to precipitate solids from a solution, but also processes involving stronger temperature reductions such as but not limited to freeze-drying. On the other hand, removal of solvent by increased temperature may also be used in the solidification process for example if the compound is in a solution or suspension at room temperatures.

As used herein, the term “demineralization” means at least a partial demineralization, preferably a removal of at least 90 mol %, more preferably at least 95 mol % of all salts.

According to the present inventive methods, first membrane filtration is carried out preferably by means of a polymeric ultrafiltration membrane having a cut-off equal to or above 4 kDa, preferably equal to or above 10 kDa and more preferably equal to or above 50 kDa, or a polymeric microfiltration membrane having a pore size of 200 nm or less, preferably 100 nm or less. Further, said second membrane filtration is preferably carried out by means of an ultrafiltration membrane having a cut-off in the range of 1 kDa to 10 kDa, preferably in the range of 2 kDa to 10 kDa and more preferably in the range of 4 kDa to 5 kDa.

The cut-off of a filtration membrane typically refers to retention of 90% of a solute of a given size or molecular mass, e.g. 90% of a globular protein with x kDa are retained by a membrane with a cut-off of x kDa. These cut-off values can be measured for example by the filtration of defined dextranes or polyethylene glycols under conditions and parameters common in the art and analysing the retentate, the permeate and the original solution, also called feed, with a GPC gel permeation chromatography analyser using methods and parameters common in the art.

As used herein, the term “cross-flow filtration” refers to a type of filtration where the majority of the feed flow travels tangentially across the surface of the filter, rather than into the filter, at positive pressure relative to the permeate side. The principal advantage of this is that the filter cake which can blind the filters in other methods is not building up during the filtration process, increasing the length of time that a filter unit can be operational. It can be a continuous process, unlike batch-wise dead-end filtration. For large scale applications, a continuous process is preferable. This type of filtration is typically selected for feeds containing a high proportion of small particle size solids where the permeate is of most value because solid material can quickly block (blind) the filter surface with dead-end filtration. According to the present disclosure, said cross-flow microfiltration or cross-flow ultrafiltration includes a cross-flow velocity in the range of 0.5 m/s to 6.0 m/s, preferably in the range of 2.0 m/s to 5.5 m/s and more preferably in the range of 3.0 m/s to 4.5 m/s. In case of a membrane made of ceramics, the cross-flow velocity may be higher than in case of a membrane made of a polymeric material depending on the respective geometry of the membrane. For example, in case of a flat polymeric membrane such as a polymeric membranes in flat sheet modules, the cross-flow velocity is 0.5 m/s to 2.0 m/s and preferably 1.0 m/s to 1.7 m/s. and more preferably 1.0 to 1.5 m/s. Depending on the particular set-up and the particular solution comprising the biomass even cross-flow velocity of 1.0 m/s or less may be used in some cases, yet the filtration may turn into a dead end filtration when the cross-flow velocity are too low. Cross-flow velocity and cross-flow speed are used interchangeably herein.

The term “cut-off” as used herein refers to the exclusion limit of a membrane which is usually specified in the form of MWCO, molecular weight cut off, with units in Dalton. It is defined as the minimum molecular weight of a solute, for example a globular protein that is retained to 90 % by the membrane. The cut-off, depending on the method, can be converted to so-called D90, which is then expressed in a metric unit.

A key part of the inventive method is the demineralisation of a solution comprising one or more fine chemicals by at least one nanofiltration step referred to as S22. This nanofiltration step S22 is preferably done with a solution comprising the fine chemical, preferably and oligosaccharide, wherein the pH of the solution has been set to a value below pH 7, preferably below pH 5, as this will make the removal of ions like phosphates or sulphates easier.

However, in a more preferred embodiment, the method involves beneficial steps before the nanofiltration step of step S22 is carried out. In this improved inventive method, in a first step (FIG. 2, step S10), a solution comprising biomass and at least one oligosaccharide is provided. Said at least one oligosaccharide comprises human milk oligosaccharide, preferably 2′-fucosyllactose. Preferably, said solution comprising biomass and oligosaccharide is obtained by cultivation of one or more types of cells in a cultivation medium. Thus, said solution may also be called fermentation broth in a preferred embodiment. The cultivation medium is preferably a cultivation medium comprising at least one carbon source, at least one nitrogen source and inorganic nutrients. More preferably, the fermentation broth or solution comprising biomass and the at least one oligosaccharide is obtained by microbial fermentation, preferably aerobic microbial fermentation. A microorganism capable of producing the oligosaccharide may be a yeast or a bacterium, for example from the group consisting of the genera Escherichia, Klebsiella, Helicobacter, Bacillus, Lactobacillus, Streptococcus, Lactococcus, Pichia, Saccharomyces and Kluyveromyces or as described in the international patent application published as WO 2015/032412 or the European patent application published as EP 2 379 708, preferably a genetically modified E. coli strain, more preferably a genetically modified E. coli strain that is deficient in the lacZ gene (lacZ-) and suitable for the production of substances for human nutrition, that is cultivated in an aqueous nutrient medium under controlled conditions, favourable for biosynthesis of the oligosaccharide, for example as disclosed in EP 2 379 708, EP 2 896 628 or US 9 944 965. The aqueous nutrient medium comprises at least one carbon source (e.g. glycerol or glucose) which is used by the microorganism for growth and/or for biosynthesis of the oligosaccharide. In addition, the nutrient medium also contains at least one nitrogen source, preferably in the form of an ammonium salt, e.g. ammonium sulphate, ammonium phosphate, ammonium citrate, ammonium hydroxide etc., which is necessary for the growth of the cells such as microorganisms. Other nutrients in the medium include e.g. one or several phosphate salts as phosphorus source, sulphate salts as sulphur source, as well as other inorganic or organic salts providing e.g. Mg, Fe and other micronutrients to the cells. In many cases, one or more vitamins, e.g. thiamine, has to be supplemented to the nutrient medium for optimum performance. The nutrient medium may optionally contain complex mixtures such as yeast extract or peptones. Such mixtures usually contain nitrogen-rich compounds such as amino acids as well as vitamins and some micronutrients.

The nutrients can be added to the medium at the beginning of the cultivation, and/or they can also be fed during the course of the process. Most often the carbon source(s) are added to the medium up to a defined, low concentration at the beginning of the cultivation. The carbon source(s) are then fed continuously or intermittently in order to control the growth rate and, hence, the oxygen demand of the cells. Additional nitrogen source is usually obtained by the pH control with ammonia (see below). It is also possible to add other nutrients mentioned above during the course of the cultivation.

In some cases, a precursor compound is added to the medium, which is necessary for the biosynthesis of the oligosaccharide. For instance, in the case of 2′-Fucosyllactose, lactose is usually added as a precursor compound. The precursor compound may be added to the medium at the beginning of the cultivation, or it may be fed continuously or intermittently during the cultivation, or it may be added by a combination of initial addition and feeding.

The cells are cultivated under conditions that enable growth and biosynthesis of the oligosaccharide in a stirred tank bioreactor. A good oxygen supply in the range of 50 mmol O2/(l*h) to 180 mmol O2/(l*h) to the microbial cells is essential for growth and biosynthesis, hence the cultivation medium is aerated and vigorously agitated in order to achieve a high rate of oxygen transfer into the liquid medium. Optionally, the air stream into the cultivation medium may be enriched by a stream of pure oxygen gas in order to increase the rate of oxygen transfer to the cells in the medium. The cultivation is carried out at 24° C. to 41° C., preferably 32° C. to 39° C., the pH value is set at 6.2 to 7.2, preferably by automatic addition of NH3 (gaseous or as an aqueous solution of NH4OH).

In some cases, the biosynthesis of the oligosaccharide needs to be induced by addition of a chemical compound, e.g. Isopropyl β-D-1-thiogalactopyranoside (IPTG) for example as in the European patent application published as EP 2 379 708. The inducer compound may be added to the medium at the beginning of the cultivation, or it may be fed continuously or intermittently during the cultivation, or it may be added by a combination of initial addition and feeding.

Subsequently, the method of the invention proceeds to the adjustment of the pH value in a second step (FIG. 2, step S12). In said step, typically the pH value of the solution is set to 7 or below. If needed it is lowered by adding at least one acid to the solution comprising biomass and the at least one oligosaccharide. Preferably, the pH value of the solution is lowered to a target pH value preferably in the range of 3.0 to 5.5, more preferably in the range of 3.5 to 5 and even more preferably in the range of 4.0 to 4.5, such as 4.0 or 4.1. Said at least one acid is an acid selected from the group consisting of H2SO4, H₃PO₄, HCl, HNO3 (preferably not in concentrated form) and CH3CO2H, or any other acid considered safe in production of food or feed; preferably the acid is selected from the group consisting of H2SO4, H₃PO₄, HCl and CH3CO2H. A mix of these acids may be used in one embodiment instead of a single of these acids.

Further, in another embodiment of the method of the invention, if the solution comprising biomass and the at least one oligosaccharide, at least one disaccharide or at least one monosaccharide already has a pH value below 7, preferably below pH 5.5, more preferably equal to or below pH 5.0 and even more preferably equal to or below pH 4.5, there will be no addition of any of these acids, and step S12 may be skipped and the methods of the invention for such solutions continues with Step S14.

The method then proceeds to the next step (FIG. 2, S14). In said step, one or more adsorbing agent is added to the solution comprising biomass and the at least one oligosaccharide. Preferably, the adsorbing agent is active carbon. Said adsorbing agent, preferably active carbon, is added in an amount in the range of 0.5% to 3% by weight, preferably in the range of 0.6% to 2.5% by weight and more preferably in the range of 0.7% to 2.0% by weight, such as 1.5%. In this respect, it has to be noted that the smaller the particles of the adsorbing agent are, the better the adsorption characteristics are. Said adsorbing agent, preferably active carbon, is added as a powder having a particle size distribution with a diameter d50 in the range of 2 µm to 25 µm, preferably in the range of 3 µm to 20 µm, for example those of 10 to 15 µm, and more preferably in the range of 3 µm to 7 µm such as 5 µm. More preferably, said adsorbing agent, preferably active carbon, is added as a suspension of the powder in water. Preferably, adding said adsorbing agent, preferably active carbon, to the solution is carried out after adding the at least one acid to the solution. Alternatively, adding said adsorbing agent, preferably active carbon, to the solution may be carried out before adding the at least one acid to the solution. With other words, the order of steps S12 and S14 may be changed and the order thereof is not fixed. Yet if the order is first setting of the pH below 7 to the desired pH value and then adding one or more adsorbing agents, preferably active carbon, will generate the best results with respect to protein removal and decolourization. In a preferred embodiment addition of the at least one acid antedates the addition of the at least one adsorbing agent, preferably active carbon.

In a preferred embodiment of the methods of the invention, the steps S12 and S14 are both performed and in the order S12 followed by S14.

The method then proceeds with first membrane filtration, preferably a micro- or ultrafiltration in a further step (FIG. 2, step S16) including a time suitable for the adhesion of colour components to the one or more adsorbing agents before the separation. The first membrane filtration is carried out so as to separate the biomass and the one or more adsorbing agents from the solution comprising the at least one oligosaccharide, at least one disaccharide and/or at least one monosaccharide, and by this removing the biomass and also reducing the colour components and protein in the resulting solution also called permeate comprising the oligosaccharides, disaccharides and/or monosaccharides. Basically, step S16 includes microfiltration or ultrafiltration. However, as there is a smooth transition between microfiltration and ultrafiltration and both can be used by the skilled artisan to the purpose of separating biomass, adsorbing agent and protein on one side and the permeate containing the bulk of the desired one or more oligosaccharides, one or more disaccharides and / or one or more monosaccharides on the other side. The filtration in step S16 may also be an ultrafiltration as an alternative to microfiltration. Said microfiltration or ultrafiltration is preferably carried out as cross-flow microfiltration or cross-flow ultrafiltration to improve membrane performance and reduce membrane abrasion. The details of the filtration in step S16 will be explained below. Said cross-flow microfiltration or cross-flow ultrafiltration includes a cross-flow speed in the range of 0.5 m/s to 6.0 m/s, preferably in the range of 2.0 m/s to 5.5 m/s and more preferably in the range of 3.0 m/s to 4.5 m/s, such as 4.0 m/s. In one embodiment the cross-flow speed is equal to or below 3.0 m/s, preferably between and including 1.0 and 2.0. One advantageous of the inventive method, use and the apparatus of the invention is that lower cross-flow speeds can be used to achieve good separation preferably of protein components of the solution from any oligosaccharides, disaccharides or monosaccharides. Thus, energy and equipment cost can be reduced, wear and tear on equipment and abrasion of the filtration membrane are also reduced. Said first membrane filtration, preferably microfiltration or ultrafiltration, is carried out at a temperature of the solution in the range of 8° C. to 55° C., preferably in the range of 10° C. to 50° C. and more preferably in the range of 30° C. to 40° C., such as 38° C. Said microfiltration or ultrafiltration is carried out by means of a ceramic or polymeric microfiltration membrane or ceramic ultrafiltration membrane having a pore size in the range of 20 nm to 800 nm, preferably in the range of 40 nm to 500 nm and more preferably in the range of 50 nm to 200 nm, such as 100 nm. Said ceramic material is or has at least one layer of at least one ceramic material selected from the group consisting of: Titanium dioxide (TiO₂), Zirconium dioxide (ZrO₂), Silicon carbide (SiC) and Aluminium oxide (Al₂O₃). Alternatively, said microfiltration or ultrafiltration is carried out by means of a polymeric ultrafiltration membrane having a cut-off equal to or above 10 kDa, preferably equal to or above 50 kDa, or a polymeric microfiltration membrane having particle size of 100 nm or less. Said polymeric material is at least one polymeric material selected from the group consisting of: polyethersulfone, polysulfone, polypropylene, polyvinylidene fluoride, polyacrylonitrile, polyvinylidene fluoride. Said first membrane filtration, preferably microfiltration or ultrafiltration, is carried out after a predetermined time after the adsorbing agent, preferably active carbon, has been added to the solution and thus ensures adhesion of colour components. Typically, the time needed for mixing of the solution with the added adsorbing agent until a homogenous distribution of the adsorbing agent, preferably active carbon, in the solution has been reached may suffice to allow for the adhesion of the colour components, yet a longer incubation time can be used to maximize this. In one embodiment, said predetermined time is at least 2 min, preferably at least 10 min and more preferably at least 20 min such as 25 min or 30 min.

In a preferred embodiment, the first membrane filtration of the inventive methods includes three steps as will be explained in further detail below. The first step includes a first diafiltration having a factor of 0.5 (amount of diafiltration water = starting amount of fermentation broth x diafiltration factor). During diafiltration, the amount of water added is identical to the amount of permeate discharged. The first step is a continuing step and the volume in the feed vessel is thus kept constant. The second step includes concentrating of the fermentation broth with the factor 2 by stopping the feed of diafiltration water and the level will decrease down to the target value (target value = volume or mass at the beginning of the fermentation broth / concentrating factor). Subsequently, the third step includes a second diafiltration. The permeates collected during these three steps are typically combined to form the permeate referred to in the tables below. By means of these three steps a lower dilution of the product within the permeate and an increased yield of ≥ 95% are realized. By increasing the factor of the second diafiltration, the yield may even be increased.

The method of the invention then proceeds with a second membrane filtration step (FIG. 2, step S18). Preferably an ultrafiltration of the solution comprising oligosaccharides, disaccharides and / monosaccharides obtained by the first membrane filtration of step S16 is carried out. In other words, an ultrafiltration of the permeate derived from the first membrane filtration in step S16 is carried out. Preferably, said second membrane filtration, preferably ultrafiltration, is carried out by means of an ultrafiltration membrane having a cut-off in the range of 1 kDa to 10 kDa, preferably in the range of 2 kDa to 10 kDa and more preferably in the range of 4 kDa to 5 kDa. In a particularly preferred embodiment, membranes with a cut-off of 4 kDa or 5 kDa are suitable. Said ultrafiltration membrane is at least partially made of a polymeric material. Said polymeric material is at least one polymeric material selected from the group consisting of: polyethersulfone, polyacrylonitrile, cellulose acetate. Said second membrane filtration, preferably ultrafiltration, is carried out at a temperature of the solution being in the range of 5° C. to 15° C., preferably in the range 8° C. to 13° C. and more preferably in the range 8° C. to 12° C., such as 10° C.

FIG. 1 displays the sequence of steps of the inventive methods with the time suitable for the adhesion of colour components to the one or more adsorbing agents before the separation shown as a separate optional step (step S15). Such a separate incubation step may be favourable when long times for sufficient adhesion of the undesired compounds to the adsorbing agent are required. Further, FIG. 2 depicts for the first membrane filtration as a step with three sub-steps; the three sub-steps of first membrane filtration being first diafiltration, concentrating and then optionally a second diafiltration. These are shown as S16/1, S16/2 and S16/3, respectively, in FIG. 2.

The inventive method then proceeds with optional decolourisation step 20 if desired, or directly with a first nanofiltration step (S22) as explained above.

The nanofiltration step may include concentrating actions and diafiltration actions, which sometimes are referred to washing. Depending on the limitations provided by the equipment set-up and the process, a diafiltration or washing action may come first, followed by a concentrating action, and optionally either the diafiltration or the sequence of the two actions may be repeated once or several times.

Preferably the concentrating action and the diafiltration action are done with the same equipment and the same membrane, but a set-up where there are different membranes for the concentrating action and for the diafiltration action is possible.

In a preferred embodiment, the nanofiltration step comprises a concentrating action followed by a diafiltration action. Preferably the CF is at least 3, more preferably is at least 5 and most preferably at least 10.

The diafiltration factor typically is not more than 10. In another preferred embodiment the DF is at least 0.5 or more, more preferably from and including 1.5 to and including 7, even more preferably in the range from 2 to 4, yet even more preferably from 2.5 to 3.5, and most preferably 2.5, 2.6, 2.7, 2.8., 2.9 or 3.0

The claimed method applies a nanofiltration membrane in the fashion described and one or more of the following benefits are provided: removes efficiently monovalent as well as divalent salts therefore no ion exchange step is necessary or, if demineralisation is still needed, the ion exchange treatment requires substantially less ion exchange resin; higher flux during the nano-filtration can be maintained compared to other membranes used for the same or similar purpose in the prior art, which reduces the operation time; the membrane applied in the claimed method is less prone to getting clogged compared to the prior art solutions; the membrane applied in the claimed can be cleaned and regenerated completely therefore can be reused without substantial reduction of its performance; if desired the nanofiltration can be done in a way that selectively and efficiently removes disaccharide, preferably lactose, from tri- or higher oligosaccharides, preferably HMOs, yielding an enriched tri- or higher oligosaccharide, preferably HMO, fraction.

In a preferred embodiment the first and optional second nanofiltration step are performed in a continuous set-up.

In another embodiment it is possible to combine the demineralisation by nanofiltration with other demineralisation methods. For example, an ion exchange step can be carried out after the first nanofiltration, as depicted by S22 and S26 in FIG. 1. Also, it is possible in the inventive method to have a second nanofiltration with a different membrane after the first nanofiltration S22. This may be done directly following the first nanofiltration (as depicted by S22 and S24) in FIGS. 1, 4 and 5. One embodiment is that in the first nanofiltration a more open membrane is used in diafiltration mode, and a subsequent second nanofiltration is done with a tighter membrane and concentration mode.

It is possible that the second nanofiltration can be done after another demineralization step for example by ion exchange which follows the first nanofiltration. Because tighter membranes can be used when using the membranes to concentrate after the ion exchange step, the product losses will be slightly lower.

Optional decolourization steps by absorbing agents may be performed as necessary before step 20, 26 or after step 26

In another embodiment the steps S10 to S26 are performed wherein instead of an at least one oligosaccharide, at least one monosaccharide, at least one disaccharide or a mixture of at least one monosaccharide, at least one disaccharide and / or at least one oligosaccharide are present in place of the at least one oligosaccharide.

A preferred embodiment is directed to an improved method for the demineralization of a solution comprising one or more fine chemical, preferably one or more HMO or one or more aroma compound, wherein the method comprises a first nanofiltration as described for step S22 followed by an optional second nanofiltration and a subsequent demineralization step, preferably by ion exchange, preferably as described for step S26 herein, wherein the demineralization of the fine chemical is improved by at least 50%, 100% or 150%, more preferably at least 200%, even more preferably at least 300% compared to the a solution that is not treated with the nanofiltration step(s) before demineralization or demineralized by other means than the inventive nanofiltration.

Such methods include the steps of:
a) Providing the solution comprising one or more fine chemical;
b) Adjusting the pH to the desired value below 7
c) A decolourisation step, preferably by the addition of an adsorbing agent, preferably active carbon,
d) An optional incubation step,
e) A first membrane filtration
f) A second membrane filtration of the permeate of the first membrane filtration,
g) Using the permeate of the second membrane filtration, in an optional decolourisation step, preferably by the addition of an adsorbing agent, preferably active carbon, followed by or replaced by a first nanofiltration step;
h) An optional second nanofiltration with the retentate of the first one,
i) Optional further processing steps of the retentate as shown in FIG. 4.

Further disclosed is a method for the purification of a fine chemical in a solution comprising the steps of optional pH setting to pH 7 or below, optional decolourisation, biomass separation by any means, followed by a sequence of one or more steps consisting of

A first nanofiltration S22, an optional second nanofiltration S24, an optional decolourisation and / or concentration step followed by an optional solidification step.

A further embodiment is to a method suitable for fine chemicals produced by fermentation or enzymatic or chemical synthesis or mixtures thereof: A method for the purification of a fine chemical in a solution comprising the steps of optional biomass separation by any means and optional pH setting to pH to the desired value, wherein these two optional steps may be performed in any order, followed by a sequence of one or more steps consisting of:

A first nanofiltration S22, an optional second nanofiltration S24, an optional decolourisation and / or concentration step followed by an optional solidification step.

In a further embodiment the invention relates to a rapid purification method (FIG. 5) suitable for fine chemicals produced by fermentation or enzymatic or chemical synthesis or mixtures thereof: The inventive method for the purification of a fine chemical in a solution comprising the steps of optional pH setting to pH to the desired value and a sequence of one or more steps consisting of:

A first nanofiltration S22, an optional second nanofiltration S24, an optional decolourisation and / or concentration step followed by an optional solidification step.

For the avoidance of doubt, any reference to the protein content of the solution or the permeate or retentate is referring to free protein in the solution / permeate / retentate, i.e. the protein found extracellularly and not the protein contained in the biomass if any. During fermentation and also subsequent handling and membrane filtrations, protein may be liberated from biomass and then be considered free protein.

For the avoidance of doubt, any reference to the at least one oligosaccharide, at least one disaccharide and / or at least one monosaccharide the solution or the permeate or retentate is referring to free the at least one oligosaccharide, at least one disaccharide and / or at least one monosaccharide in the solution / permeate / retentate, i.e. the at least one oligosaccharide, at least one disaccharide and / or at least one monosaccharide found extracellularly and not the ones contained in the biomass if any. During fermentation and also subsequent handling and membrane filtrations, the at least one oligosaccharide, at least one disaccharide and / or at least one monosaccharide may be liberated from biomass and then be considered free the at least one oligosaccharide, at least one disaccharide and / or at least one monosaccharide in the solution.

In a preferred embodiment, the step of carrying out first membrane filtration, preferably a microfiltration or ultrafiltration, so as to separate the biomass from the solution comprising the at least one oligosaccharide, at least one disaccharide and / or at least one monosaccharide is to be understood as a step of separating the biomass from the at least one oligosaccharide, at least one disaccharide and / or at least one monosaccharide, wherein the majority of the at least one oligosaccharide, at least one disaccharide and / or at least one monosaccharide is found in the permeate of the first membrane filtration following the separation of biomass.

In a preferred embodiment, the first membrane filtration is followed by an ultrafiltration, then optionally followed by a nanofiltration or reverse osmosis, preferably a nanofiltration, followed by ion exchange.

If following step S26 or S24 a reverse osmosis is performed to achieve at least some purification rather than just concentration of the solution, in one embodiment this is considered a nano-filtration within the meaning of the present application, and hence the inventive method would include as a nanofiltration step a membrane and set-up that could also be used for reverse osmosis.

Also, the nanofiltration of step 22 can be replaced by a reverse osmosis when a concentration of the solution rather than a purification is desired and then combined with second nanofiltration step S24 preferably including at least one defiltration sub-step.

In another embodiment, a method for the purification of one or more HMO(s) comprising the steps of:
i. providing a solution comprising biomass and one or more fine chemical,
ii. setting the pH value of the solution below 7, preferably below pH 5.5 or less by adding at least one acid to the solution comprising biomass and the at least one oligosaccharide,
iii. adding an adsorbing agent to the solution comprising biomass and fine chemical,
iv. Optionally an incubation step,
v. carrying out a membrane filtration also called herein the first membrane filtration and typically being a microfiltration or ultrafiltration so as to separate the biomass from the solution comprising the at least one fine chemical;
vi. Optionally carrying out at least one second or further membrane filtration with the permeate of the first membrane filtration, preferably at least one ultrafiltration;
vii. Demineralizing the solution with one or more nanofiltration(s);
viii. Completing the purification of the HMO without any further demineralisation like ion exchange steps,
is disclosed.

In a preferred embodiment the inventive method is a method for the improved purification of neutral or acidic human milk oligosaccharides, more preferably for the purification of 2′-fucosyllactose, LNT and 6′SL alone or in combination.

In another preferred embodiment the method of the invention is used to purify a solution comprising one or more aroma compound(s) instead of or in addition to oligosaccharides, disaccharides or monosaccharides.

Preferably, the nanofiltration steps of the methods of the invention are performed as crossflow nanofiltration with spiral wound membranes.

One embodiment is directed to a method for separating a oligosaccharide and / or disaccharide from salts which are dissolved in a feed solution, particularly in an aqueous medium from a fermentation or enzymatic process, comprising: a) contacting the feed solution with a nanofiltration membrane with a molecular weight cut-off ensuring the retention of the oligosaccharide and the disaccharide and allowing at least a part of the salts to pass, wherein membrane is a thin-film composite membrane of which the active (top) layer of the membrane is composed of e.g. polyamide, and wherein the NaCl retention of the membrane is less than 30%, preferably less than 20%, more preferably less than 15% and even more preferably less than 10%, b) a subsequent optional diafiltration with said membrane, and c) collecting the retentate enriched in the oligosaccharide and / or disaccharide.

Summarizing, the present invention includes the following embodiments, wherein these include the specific combinations of embodiments as indicated by the respective interdependencies defined therein.

### Further Embodiments

1. Method for purification of one or more oligosaccharides from a solution comprising biomass and one or more oligosaccharides, comprising the steps of
i. providing a solution comprising biomass and one or more fine chemical, preferably oligosaccharide or aroma compound,
ii. setting the pH value of the solution below 7, preferably below pH 5.5 or less by adding at least one acid to the solution comprising biomass and the at least one oligosaccharide,
iii. decolourizing the solution at least in part by adding an adsorbing agent to the solution comprising biomass and fine chemical, preferably oligosaccharide or aroma compound,
iv. Optionally an incubation step,
v. carrying out a membrane filtration also called herein the first membrane filtration and typically being a microfiltration or ultrafiltration so as to separate the biomass from the solution comprising the at least one fine chemical, preferably oligosaccharide or aroma compound;
vi. Optionally carrying out at least one second or further membrane filtration with the permeate of the first membrane filtration, preferably at least one ultrafiltration;
vii. Optionally carrying out a decolourization step
viii. Carrying out a nanofiltration with the permeate of the membrane filtration antedating this step viii, either with the permeate of the first or the second or any further membrane filtration;
ix. Optionally a decolourization step;
x. Optionally a second nanofiltration with the retentate of the nanofiltration of the previous nanofiltration step viii, wherein the nanofiltration membrane used is a different one to the one in the previous nanofiltration step;
xi. Optionally a third or further nanofiltration with a membrane differing from the one of the previous nanofiltration step;

2. The method of embodiment 1 with subsequent to step xi further processing of the retentate of the previous nanofiltration step by any of the following steps are conducted preferably in this order:
a. carrying out a decolourization step, and / or
b. carrying out a demineralization step, more preferably a cation exchange and / or anion ion exchange, and / or
c. carrying out an electrodialysis and / or reverse osmosis and / or concentration step and / or a decolourization step; and / or
d. carrying out a simulated moving bed chromatography, and / or a solidification step creating a solid fine chemical, preferably oligosaccharide or aroma compound product, preferably a crystallisation step and / or a spray drying of the fine chemical, preferably oligosaccharide or aroma compound followed by drying as desired.

3. The method according to embodiment 2 wherein a demineralization step is conducted after the last one of the one or more nanofiltrations of steps viii, x and xi, wherein the demineralization is performed by ion exchange and wherein further the throughput of the demineralisation step is increased preferably by a factor of at least 2, more preferably 2.5, 3.0, 3.5, 4.0, 4.25 or 4.5 or more compared to the throughput in the ion exchange step without the one or more nanofiltrations of steps viii, x and xi.

4. A method for the demineralization of a solution comprising one or more oligosaccharides, preferably one or more HMO, wherein the method comprises the steps of:
a. Providing the solution comprising one or more oligosaccharides;
b. Preferably adjusting the pH to the desired value below 7, preferably below pH 5.5 or less by adding at least one acid to the solution comprising at least one oligosaccharide,
c.A preferred decolourisation step, preferably by the addition of an adsorbing agent, preferably active carbon,
d. An optional incubation step,
e. carrying out a first membrane filtration and preferably being a microfiltration or ultrafiltration
f. A second membrane filtration of the permeate of the first membrane filtration,
g. optional decolourisation step of the permeate of the second membrane filtration, preferably by the addition of an adsorbing agent,
h. a first nanofiltration step; with a sub-step of concentration and/or a sub-step of diafiltration, preferably a sub-step of concentration and a sub-step of diafiltration, more preferably a first sub-step of concentration followed by a second sub-step of diafiltration.

5. The method according to embodiment 4, wherein the concentration sub-step of step h) is performed so that the concentration factor is at least 3, preferably at least 3.5 to 10, and more preferably 10 or more.

6. The method according to embodiments 4 or 5, wherein the diafiltration sub-step of step h) is performed so that the diafiltration factor is around 3.

7. The method of any of embodiments 4 to 6, wherein the demineralization of the solution comprising the one or more oligosaccharide is improved by at least 150%, more preferably at least 200%, even more preferably at least 300% compared to the a solution that is not treated with the nano-filtration step(s) before demineralization or demineralized by other means than nanofiltration according to step e and optionally f.

8. Any of the methods of the previous embodiment, wherein the nanofiltration membrane has a NaCl retention of the membrane between 5 and 30%, preferably between 5 and 20%, more preferably between 5 and 15% and even more preferably between 5 and 10%.

9. The method according to any one of the preceding embodiments, wherein the pH value of the solution is lowered to a pH value in the range of 3.0 to 5.5, preferably the range of 3.5 to 5 and more preferably the range of 4.0 to 4.5.

10. The method according to any one of the preceding embodiments, wherein said at least one acid is an acid selected from the group consisting of H₂SO₄, H₃PO₄, HCI, HNO₃ and CH₃CO₂H.

11. The method according to any one the preceding embodiments, wherein said adsorbing agent, preferably active carbon, is added in an amount in the range of 0.5% to 3% by weight, preferably in the range of 0.75% to 2.5% by weight and more preferably in the range of 1.0% to 2.0% by weight.

12. The method according to any one of the preceding embodiments, wherein said adsorbing agent, preferably active carbon, is added as a powder having a particle size distribution with a diameter d50 in the range of 2 µm to 25 µm, preferably in the range of 3 µm to 20 µm and more preferably in the range of 3 µm to 7 µm or 10 µm to 15 µm.

13. The method according to any one of the preceding embodiments, wherein said first membrane filtration is carried out as cross-flow microfiltration or cross-flow ultrafiltration.

14. The method according to embodiment 13, wherein said cross-flow microfiltration or cross-flow ultrafiltration includes a cross-flow speed in the range of 0.5 m/s to 6.0 m/s, preferably in the range of 2.0 m/s to 5.5 m/s and more preferably in the range of 3.0 m/s to 4.5 m/s.

15. The method according to embodiment 14, wherein said cross-flow speed is equal to or below 3 m/s and preferably for polymeric membranes equal to or below 1.7 m/s

16. The method according to any one of the preceding embodiments, wherein said first membrane filtration is carried out at a temperature of the solution in the range of 8° C. to 55° C., preferably in the range of 10° C. to 50° C. and more preferably in the range of 30° C. to 40° C.

17. The method according to any one of the preceding embodiments, wherein said first membrane filtration is carried out by means of a ceramic microfiltration or ultrafiltration membrane having a pore size in the range of 20 nm to 800 nm, preferably in the range of 40 nm to 500 nm and more preferably in the range of 50 nm to 200 nm, or wherein said first membrane filtration is carried out by means of a polymeric ultrafiltration membrane having a cut-off equal to or above 10 kDa, preferably equal to or above 50 kDa, or a polymeric microfiltration membrane having a pore size of 100 nm or less.

18. The method according to any one of the preceding embodiments, further comprising carrying out a second membrane filtration with the solution comprising oligosaccharides obtained by the first membrane filtration, preferably an ultrafiltration with a membrane having a lower cut-off than the membrane of the first membrane filtration.

19. The method according to embodiment 18, wherein said second membrane filtration is an ultrafiltration and is carried out by means of an ultrafiltration membrane having a cut-off in the range of 1 kDa to 10 kDa, preferably in the range of 2 kDa to 10 kDa and more preferably in the range of 4 kDa to 5 kDa.

20. The method according to any one of embodiments 18 or 19, wherein said second membrane filtration is carried out at a temperature of the solution being in the range of 5° C. to 15° C., preferably in the range 8° C. to 13° C. and more preferably in the range 8° C. to 12° C.

21. The method according to any one of the preceding embodiments, wherein said at least one oligosaccharide comprises human milk oligosaccharide, preferably 2′-fucosyllactose, 6′-sialyllactose and/or Lacto-N-tetraose, more preferably 2′-fucosyllactose.

### Embodiment 22:

A method for separating an oligosaccharide and / or disaccharide from salts which are dissolved in a feed solution, particularly in an aqueous medium from a fermentation or enzymatic process, comprising:
i. contacting the feed solution with a nanofiltration membrane with a molecular weight cut-off ensuring the retention of the oligosaccharide and / or the disaccharide and allowing at least a part of the salts to pass, wherein the NaCl retention of the membrane is less than 30%, preferably less than 20%,
ii. a subsequent optional diafiltration with said membrane, and
iii. collecting the retentate enriched in the oligosaccharide and / or disaccharide.

### Embodiment 23:

The method according to any of the embodiments 22, wherein the top layer of the membrane is composed of polyamide.

### Embodiment 24:

The method according to any of the embodiment 23, wherein the polyamide nanofiltration membrane is a thin-film composite (TFC) membrane.

### Embodiment 25:

The method according to any of the embodiments 22 to 24, wherein the pure water flux of the membrane is at least 3 L/(m2.h.bar).

### Embodiment 26:

The method according to any of the embodiments 22 to 25, wherein the polyamide nanofiltration membrane is a piperazine-based polyamide membrane.

### Embodiment 27:

The method according to any of the embodiments 22, wherein the active layer is a polyelectrolyte multilayer.

### Embodiment 28:

The method according to any of the embodiments 22 to 27, wherein the said tri- or higher oligosaccharide comprises said disaccharide in its structure.

### Embodiment 29:

The method according any of the embodiments 22 to 28, wherein said disaccharide is lactose.

### Embodiment 30:

The method according to embodiment 28, wherein the tri- or higher oligosaccharide is a human milk oligosaccharide (HMO), preferably a tri- to octasaccharide HMO.

### Embodiment 31:

The method according to embodiment 30, wherein the HMO is a neutral HMO.

### Embodiment 32:

The method according to embodiment 31, wherein the neutral HMO is a fucosylated HMO, preferably 2′-FL, 3-FL, DFL or LNFP-I.

### Embodiment 33:

The method according to embodiment 31, wherein the neutral HMO is a non-fucosylated HMO, preferably lacto-N-triose II, LNT, LNnT, pLNnH or pLNH II.

### Embodiment 34:

The method according to embodiment 30, wherein the HMO is a sialylated HMO, preferably 3′SL or 6′-SL.

### Embodiment 35:

The method according to any of the embodiments 30 to 34, wherein the HMO is produced by fermentation or enzymatically from lactose as precursor.

### DESCRIPTION OF FIGURES

FIG. 1 shows a block diagram of the method for purification of one or more fine chemicals from a solution comprising for example biomass and at least one fine chemical according to the present invention. Dashed outlines indicate optional parts, whereas, dotted outlines indicate parts which are, in case of demineralization according to claim 1, optional but otherwise non-optional. S10 denotes the provision of the solution comprising the fine chemical, preferably an HMO or an aroma compound; S12 is adjusting the pH value to the desired pH preferably below pH 7; S14 is a decolourization step; S15 is an optional incubation step with the adsorbing agent; S16 a first membrane filtration (MF) step; S18 a second membrane filtration step; S20 is a decolourization step; S22 a first nanofiltration (NF) step; S24 a second nanofiltration step,; S26 an optional demineralization step, shown typically as an ion exchange step (IEX); Decol.; Conc.; ED; RO indicates optional Decolourization, Concentration, Elektrodialysis and / or Reverse Osmosis steps in any order; SMB is short for simulated moving bed chromatography; Solidification indicates the step of producing solid particles of fine chemical if desired - some applications may prefer the fine chemical product to be in a purified solution instead. Perm. Stand for permeate; Ret. Stands for retentate; Reg. stands for regenerate of the ion exchanger; FT stand for the flow-through of the ion exchanger that largely comprises the desired fine chemical(s)

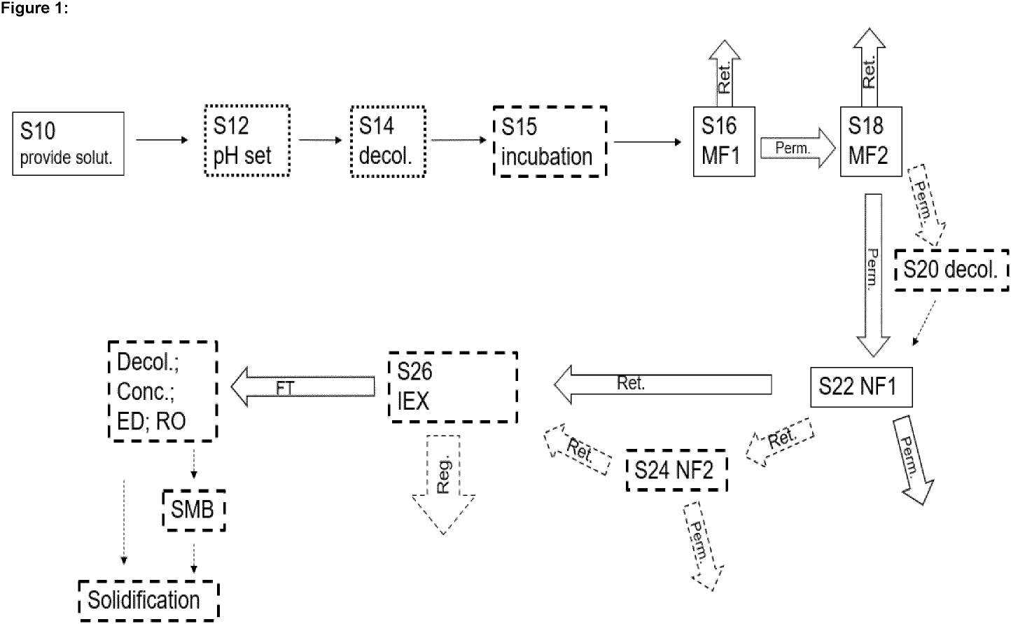

FIG. 2 shows in a block diagram a more preferred method for purification of oligosaccharides or other fine chemicals. Abbreviations, depictions and steps are as shown in FIG. 1, with the following changes: S10 is the provision of a solution comprising the fine chemical, preferably the oligosaccharide, and biomass in form of a fermentation broth. The first membrane filtration S16 is shown in three sub-steps (S16/1 to S16/3) of first membrane filtration being first diafiltration DF, concentrating C. and then optionally a second diafiltration. The second membrane filtration S18 is preferably an ultrafiltration (UF); Step 22 is shown in more detail as a first concentration sub-step (S22/1) of the nanofiltration followed by a second sub-step in diafiltration mode (S22/2). The demineralisation step by ion exchange is shown in two sub-steps, first (S26/1) a cation exchanger resin (CIEX) is used, preferably a strong one, and a subsequent sub-step S26/2 with an anion exchange resin (AlEX), preferably a weak one; Following step S26 in the preferred method, no further decolourisation is needed.

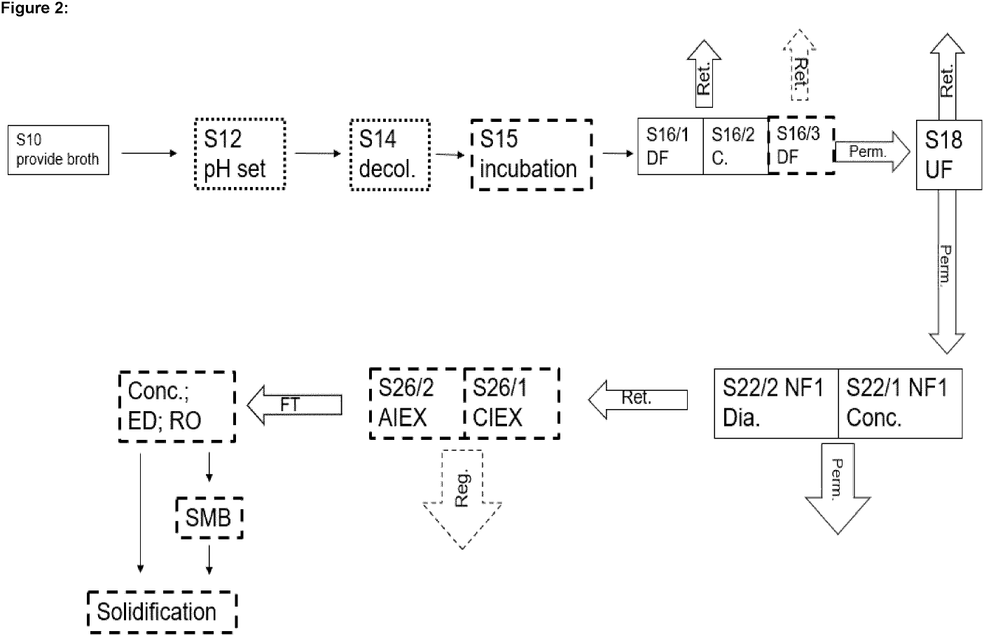

FIG. 3 shows the schematic set-up of a unit for testing the nanofiltration using spiral wound membrane elements. The unit is equipped with two pumps, one for feed pressure and one to generate the desired crossflow.

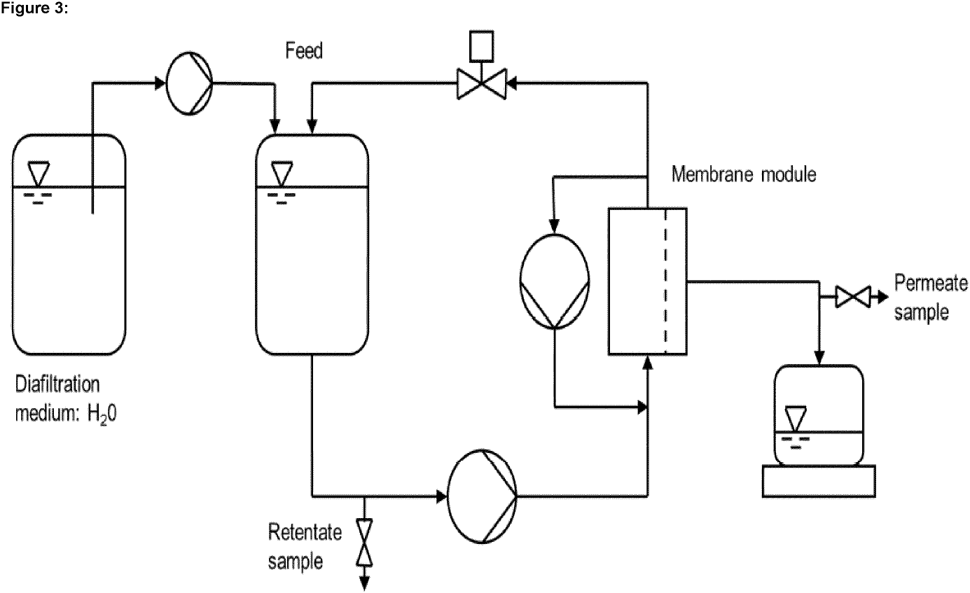

FIG. 4 shows as block diagram another preferred method for the purification of fine chemicals from fermentation broth. After the provision of the fermentation broth S10 it continuous in the same manner as in FIG. 1 but there is no demineralization step S26 included any longer, as the combination of biomass separation and nanofiltration to remove the ions results already in a purified solution that is suitable for use or the optional further processing steps as shown in FIG. 4.

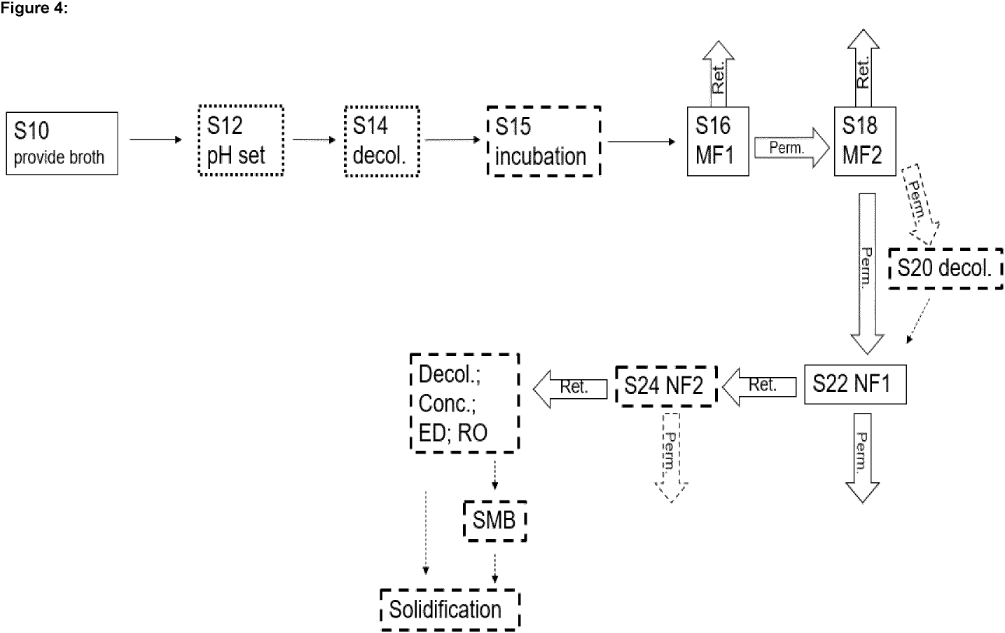

FIG. 5 shows as block diagram another preferred method for a rapid purification of a fine chemical such as an oligosaccharide from a solution comprising said fine chemical, wherein the method begins with an optional step of adjusting the pH to the desired value below 7, then a first and an optional second membrane filtration step as described herein as S22 and S24 respectively, and optional further processing steps of the purified solution.

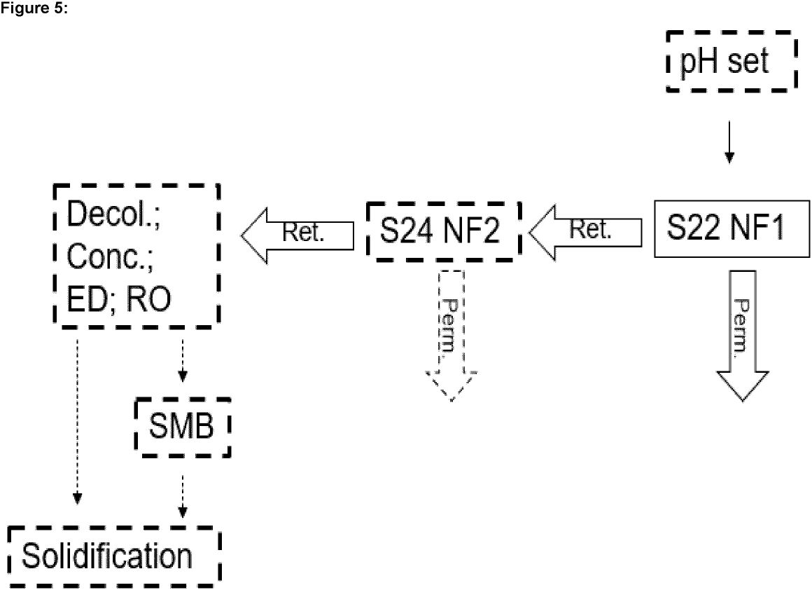

### EXAMPLES

The method according to the present invention will be described in further detail below. Whatsoever, the Examples shall not be construed as limiting the scope of the invention.

### Analytical Methods

The following analytical methods have been carried out.
HPLC for the determination of the product, i.e. human milk oligosaccharides, disaccharides, monosaccharides, and secondary components
Drying balances for measuring the dry content
APHA for measuring the colour using standard methods, for example DIN EN ISO 6271
Bradford protein assay for measuring the concentration of protein.

### Abbreviations and Symbols

Hereinafter, the following abbreviations are used:
AC = Active Carbon
UF = Ultrafiltration
NF = Nanofiltration
DP = Pressure drop along the module (p_{feed} - pᵣₑₜₑₙₜₐₜₑ)
Cross-flow velocity = linear speed of the suspension in membrane channels (m/s)
Membrane load = amount of permeate produced by 1m2 of membrane area (m3/ m2)

Further, regarding the liquid separation, the following symbols and explanations are used.

| Symbol | Meaning | Unit | Definition |
|---|---|---|---|
| Letters | | | |
| CF | Concentration factor | - | m_{R,t=0}/ m_{R} |
| DF | Diafiltration factor | - | m_{P}/m_{R,t=0} |
| J | Flux | LMH = L m⁻² h⁻¹ | |
| P | Permeance, some-times also referred to as permeability | L m⁻² h⁻¹ bar⁻¹ | |
| m | Mass | kg | |
| p | Pressure | bar | |
| R | Retention | - | 1 - cₚₑᵣₘₑₐₜₑ/cᵣₑₜₑₙₜₐₜₑ |
| TMP | Trans-membrane pressure | bar | (p_{feed} + pᵣₑₜₑₙₜₐₜₑ)/2 - pₚₑᵣₘₑₐₜₑ |

The retention for a specific compound i is calculated by:

$${R}_{i} = 1 - \frac{{c}_{i}^{\text{permeate}}}{{c}_{i}^{\text{retentate}}}$$

i.e one minus the ratio of the concentration of a component i in the permeate to the concentration of a component i in the retentate.

When a mixture is diafiltrated, the concentration C of a component i decreases exponentially with the diafiltration factor DF according to the following relation:

$${C}_{i} = {C}_{i}^{0} exp \left(- \left(1 - {R}_{i}\right) ⋅ D F\right)$$

with Cᵢ⁰ being the concentration of the compound I at time 0.

### Initial Purification Steps - Decolourization, Removal of Biomass and Initial Membrane Filtrations

A fermentation broth as a complex solution comprising biomass and at least one oligosaccharide has been prepared by standard methods. The pH value thereof has been lowered to 4 ± 0.1 by means of adding 10% sulfuric acid. Thereafter, about 100 g or more per 2.5 kg complex solution of a 30% suspension of active carbon Carbopal Gn-P-F (Donau Carbon GmbH, Gwinner-strasse 27-33, 60388 Frankfurt am Main, Germany), which is food safe, has been added and stirred for 20 min.

The thus prepared solution has been supplied to the process apparatus, a semi-automatic MF lab unit from Sartorius AG, Otto-Brenner-Str. 20, 37079 Goettingen, Germany, modified for the purpose, and heated to 37° C. in a circulating manner with closed permeate. For separation purposes, the process apparatus included a ceramic mono channel element (from Atech Innovations GmbH, Gladbeck, Germany) having an outer diameter of 10 mm, an inner diameter of 6 mm, a length of 1.2 m and a membrane made of Al₂O₃ having a pore size of 50 nm. As soon as the circulation of the solution is running and the solution comprises the target temperature of 37° C., the discharging of the permeate has been started and the control of the trans membrane pressure has been activated.

After terminating of the inventive method, the process apparatus has been stopped, the concentrate has been disposed and the process apparatus has been cleaned. Cleaning has been carried out by means of 0.5% to 1% NaOH at a temperature of 50° C. to 80° C., wherein the NaOH has been subsequently removed by purging.

i) With a fermentation broth containing inter alia 2-fucosyl lactose (2-FL), only a first diafiltration step with DF =1 and a concentrating step with CF = 2 were performed, each by means of a 50 nm Al₂O₃ membrane (available from Atech Innovations GmbH, Germany) and at a temperature of 40° C., a transmembrane pressure (TMP) of 1.2 bar and a cross-flow velocity of 4 m/s. Then, the first membrane filtration was stopped, the resulting solutions and remainder of the starting solutions were analyzed and the results compared.

Table A shows the analytical results depending on the pH value and active carbon. DC is the abbreviation for dry content. OD for the optical density.

TABLE A

| pH | Sample | DC | APHA | OD | 3.2-Di-Fl | 2FL | 2F-Lactulose | Lactose | Protein |
|---|---|---|---|---|---|---|---|---|---|
| | | [%] | | | [g/l] | [g/l] | [g/l] | [g/l] | [g/l] |
| 7.0 | Feed | 17.8 | | 138 | 3.43 | 62.07 | 0.6 | 4.28 | 0.478 |
| | Permeate | 7.61 | 4196 | | 1.99 | 34.54 | 0.43 | 2.54 | 0.124 |
| | Concentrate | 18.5 | | 136 | | | | | 1.882 |
| 7.0+1% AC | Feed | 18.3 | | 119 | 3.29 | 62.22 | 0.33 | 3.93 | 0.964 |
| | Permeate | 7.9 | 1467 | | 2.14 | 37.69 | 0.26 | 2.25 | 0.073 |
| | Concentrate | 17.3 | | 237 | 1.89 | 31.39 | 0.54 | 0.12 | 1.41 |
| 4.0 | Feed | 17.3 | | 150 | 2.83 | 54.98 | 0.59 | 0.89 | 0.76 |
| | Permeate | 8.2 | 4784 | | 1.90 | 35.00 | 0.37 | 2.57 | 0.019 |
| | Concentrate | 17.7 | | 434 | | | | | 0.026 |
| 4.0+1%AC | Feed | 16.8 | | 151 | 2.83 | 55.52 | 0.34 | 3.65 | 0.760 |
| | Permeate | 7.8 | 781 | | 1.73 | 33.66 | 0.27 | 2.26 | 0.019 |
| | Concentrate | 18.3 | | 293 | 1.61 | 29.14 | 0.33 | 2.38 | 0.026 |

The following results are derivable from Table A: Adding 1% active carbon to the fermentation broth reduces the color value of the permeate. At a pH value of 7, 1% active carbon reduces the color value at approximately 65%. At a pH value of 4, 1% active carbon reduces the color value at approximately 84%. Thus. the color value is below the upper end of 1000 and a further decolorization is not necessary. Adding active carbon at a pH value of 7 reduces the concentration of protein within the permeate at approximately 40%. whereas no effect in this respect by adding active carbon can be derived at a pH value of 4 over the pH effect on protein concentration. Nevertheless. the concentration of protein within the permeate at a pH value of 4 and with adding 1% active carbon is smaller by a factor of 4 if compared to the concentration of protein within the permeate at a pH value of 7 and with adding of 1% active carbon. Adding active carbon has no significant influence on the concentration of the oligosaccharides 3.2-Di-fucosyllactose (3.2-Di-Fl). 2′Fucosyllactulose (2F-Lactulose) and 2′Fucosyllactose (2FL). within the permeate at both pH values. Thus. it can be derived that these components do not adhere to the active carbon in significant amounts. The disaccharide lactose shows in this experiment a small reduction in concentration when active carbon is used. yet the beneficial effect of lowered pH and active carbon allow for the application of the inventive method for this disaccharide.

ii) Several batches of fermentation broths produced with standard methods comprising 6′-sialyllactose, or Lacto-N-tetraose, have been subjected to the inventive methods. Lowering of the pH value and decolourization with an absorbing agent were the first steps.

First, the steps S10 to S18 were performed. Fermentation broths comprising Lacto-N-tetraose starting with a high concentration of colour components resulting in APHA values of 7000 or more in the fermentation broth, gave permeates after the first membrane filtration - by means of a 50 nm Al₂O₃ membrane (available from Atech Innovations GmbH, Germany) and at a temperature of 40° C., a transmembrane pressure (TMP) of 1.2 bar and a cross-flow velocity of 4 m/s -with an APHA value of below 1000, but typically below 300. The protein concentration was lowered from typically around 3 g/l to less than 0.01 g/l. The vast majority, typically above 95 %, of the Lacto-N-tetraose originally found in the fermentation broth was present in the combined permeate. Similarly, for other oligosaccharides present and also for the disaccharide lactose most was present in the combined permeate and only minor amounts found in the retentate at the end of the first membrane filtration. The applied DF values were below or equal to 3.5. For 6-SL and LNT, first a set-up with a diafiltration factor of 3 followed by a set-up with a concentration factor of 2 proved useful.

Also, fermentation broths comprising 6′-sialyllactose with APHA values of around 7000, after said first membrane filtration resulted in permeates with an APHA value of below 300. The protein concentration was lowered by a factor of at least 10 or more, even by more than 100, compared to the starting value in the fermentation broth, at DF values below 3. The vast majority, typically above 90% of the 6′-sialyllactose originally found in the fermentation broth was present in the combined permeate. Similarly, for other oligosaccharides present and also for the disaccharide lactose most was present in the combined permeate and only minor amounts found in the retentate at the end of the first membrane filtration.

It was also found that performing the methods with a pH of below 5.5 improved flux in the first membrane filtration compared to higher pH values (cross-flow speed 3.5 m/s, temperature 30° C.; DF = 3). This improved even further when the pH value of the solution comprising the biomass and the 6′- sialyllactose was pH 4.2. Compared to pH 6.3, the flux more than doubled when pH 5.2 was used and tripled when the pH value was pH 4.2.

The combined permeate of the first membrane filtration were submitted to an ultrafiltration as second membrane filtration.

For both 6′SL and LNT, the 4 kDa PES polymeric membrane (50 nm) UH004 of MICRODYN-NADIR GmbH, Kasteler Strasse 45, Gebaude D512, 65203 Wiesbaden/Germany gave a good performance with high performance and hardly any fouling. For 6′SL the pH of the fermentation broth was set to pH 4 with 10 to 20% sulphuric acid or phosphoric acid, 1.4% (w/w) active carbon were mixed in, and the first membrane filtration was conducted at 30-37° C., velocity 3.5 m/s: DF1=3 to 3.5 followed by CF=2 with a yield ≥95% at this first membrane filtration. Then as second membrane filtration an ultrafiltration was done with a 4 kDa PES Membrane at 8-12° C. and 10 bar, Cross-flow velocity of 1.5 m/s, with a CF1 >10 (up to 20), followed by a DF ≥ 3; the total yield was 95%.

For LNT, the pH of the fermentation broth was set to pH 4 with 10 to 20% sulphuric acid or phosphoric acid, 1.0% (w/w) active carbon were mixed in and stirred for 50 minutes, and the first membrane filtration was done with the Al₂O₃ membrane (50 nm) membrane was conducted at 30-37° C. with 3.5 m/s and a DF of 3, followed by a CF of 2; the yield was >97%. The following second membrane filtration was an ultrafiltration similar to the one for 6′SL at 8-12° C. and 10 bar with a concentration factor up to 80 and a subsequent DF = 3; total yield was over 99%.

The resulting permeates were stored refrigerated prior to use in the first nanofiltration or, if stored for longer times, frozen thawed and agitated before the first nanofiltration.

### Nanofiltration

A number of nanofiltration membranes were tested with different oligosaccharides.

TABLE 1

Overview of the used membranes and supplier’s cut-off and/or retention indications

| | | |
|---|---|---|
| Membrane | Supplier | Retention / Cut-Off |
| TS40 | | 40% NaCl |
| | | 99.0% MgSO₄ |
| TS80 | | 80% NaCl |
| | | 99.2% MgSO₄ |
| UA60 | Microdyn-Nadir (Trisep) | 10% NaCl |
| | | 80% MgSO₄ |
| XN45 | | 20% NaCl |
| | | 96% MgSO₄ |
| NP030 | | 80-95% Na₂SO₄ |
| ESNA 3J | | 0.15-0.25 kD |
| 7450 | Nitto-Denko | 50% NaCl |
| 7470 | | 70% NaCl |
| Desal DL | SUEZ | 96% MgSO₄ |
| AS3014 | AMS Technologies | 0.4 kD |
| | | >92% MgSO₄ |
| dNF40 | NX Filtration | 0.4 kD |
| dNF80 | | 0.8 kD |

Most of the tested membranes showed very good retention of the oligosaccharide and often lactose as well. However, the tests also showed that some membranes are not well suited to let salts and other ions like phosphoric acid pass. Others, like UA60 or XN45 or AS3014 showed encouraging results indicated that separation of oligosaccharides from the ions like phosphoric acid can be achieved. Depending on the oligosaccharide and the set-up, less than 8%, less than 27% or less than 52% of the phosphoric acid was found in the retentate, respectively. The TS80-membrane shows a very high retention for LNT. In an experiment using the same set-up starting from the provision of the fermentation broth to a nanofiltration with the permeate of the ultrafiltration as second membrane filtration, but using a fermentation broth from bacterial strain producing the neutral HMO 2′-Fucosyllactose, the TS80 membranes showed retention values of >99% for the smaller 2′-FL molecule. However, TS80 also showed a strong retention of phosphoric acid as well.

The hollow fibre dNF 40 (not shown in the table above) was tested for 6′SL only and showed a retention of this HMO of 99.1 % while only 62.6 % of phosphoric acids was retained in this test.

### Spiral Wound Elements

Spiral wound elements of nanofiltration membranes allow for a better scalability to large scale processes than for example experiments with flat-sheet membranes.

Experiments ran in crossflow set-up with spiral-wound elements, see Table 2. The first two experiments termed 002 and 003 used the UA60 membrane for concentrating with a CF of 10 and in case of experiment 003 for diafiltration with a DF of 2.9. Washing indicates that de-ionized water was added to the retentate continuously in the same amounts as permeate was removed. Experiments 006 and 007, were done similar to experiments 002 and 003 but to check the performance of the membrane at a lower concentration factor.

TABLE 2

Overview of the LNT-experiments with spiral-wound elements

| Experiment | Membrane | Goal | Process variant |
|---|---|---|---|
| 002 | UA60 | Concentrating UF-permeate by CF = 10.0 | NF before IEX |
| 003 | UA60 | Concentrating UF-permeate by CF = 10.9, followed by washing with DF = 2.9 | NF before IEX |
| 006 | UA60 | Concentrating UF-permeate by CF = 7.6 | NF |
| 007 | UA60 | Concentrating UF-permeate by CF = 7.6, followed by washing with DF = 3.0 | NF |

Table 3 gives an overview of the purification by the nanofiltration steps. The purification is given in terms of product retentions for LNT, lactose and phosphoric acid (HPLC-analysis), since these parameters can be scaled for other concentration or diafiltration factors.

TABLE 3

Overview of the membrane retentions and changes in conductivity

| | | | | | |
|---|---|---|---|---|---|
| Experiment¹ (...) | Membrane | R_{LNT} (%)¹ | R_{lactose} (%)¹ | R_{H3PO4} (%)¹ | Conductivity² (mS/cm) |
| 002 | UA60 | R: 92.4 | R: 75.4 | R: 19.6 | 6.4 → 8.02 |
| | | P: 99.3 | P: 80.0 | P: 24.6 | |
| 003-CF | UA60 | R: n/a P: 99.3 | R: n/a P: 75.6 | R: n/a P: 17.4 | 6.4 → 3.77 |
| 003-DF | | P: n/a | n/a | n/a | |
| 006 | UA60 | R: 86.1 P: 99.8 | R: 87.6 P: 94.8 | R: 8.0 P: 26.4 | n/a |
| 007-CF | UA60 | R: n/a P: 98.5 | R: n/a P: 76.9 | R: n/a P: 23.9 | n/a |
| 007-DF | | P: n/a | P: n/a | P: n/a | |

¹ R indicates calculation over the retentate, P calculation over the permeate
² Feed → Final concentrate (i.e. after diafiltration, when applicable)

As can be seen in Table 3, the retention of the UA60 membranes for LNT is generally high (>98.5% is measured for all data, based on the permeate, in many cases >99% was measured). Simultaneously, lactose retentions of 75-95% were recorded, washing led to lower lactose retention. The exact value varied depending on the conditions applied in this test. For the phosphoric acid, very low retentions in the range of 15-25% were measured. These data correspond to the measurement in the test cells, where similar values were recorded.

An overview of yields in the final retentates of the experiments in comparison with the feed for these experiments was as follows:

The experiments showed that with the UA60 membrane the yields of LNT in the retentate was good. Lactose yields in the retentate were nearly as high, but the phosphate was largely eliminated with the permeate. If concentration and diafiltration mode was used, the overall yield of LNT was good as well, but in contrast lactose yield was much lower. Hence, by choosing the set-up of the nanofiltration one can steer whether LNT and lactose are both retained, or if the HMO is preferably retained and lactose is reduced in comparison to the LNT. Phosphate was even better removed in this type of nanofiltration and only very small amounts of it were found in the retentates.

Experiments 002 and 003 were selected for demineralization after the nanofiltration step.

### Test With Flat-sheet Membranes for 6′-SL

Three test cell experiments were performed with UF permeates of different pHs. All experiments ran a diafiltration with DF = 3 followed by a concentration step with CF = 10. This sequence is not fully optimized but should show the potential of removing salts and potentially smaller molecules using nanofiltration. Higher removal rates can possibly be reached when higher diafiltration factors are applied.

Table 4 presents an overview of the results. Independent of the feed pH, all experiments succeeded in the removal of acetic acid to below the detection limit. The phosphate levels relative to 6SL were reduced dramatically in the retentate at all measured pH values, and at pH5.56 there was an absolute reduction of phosphates in the retentate of significance as well.

One of the most interesting removals was the removal of lactose, since this could significantly ease the downstream crystallization or SMB step. In the experiments at pHs of 4.4 and 5.56, about half of the lactose was removed through the current way of running the process. Using a higher diafiltration factor, it can be expected that some more lactose could be removed. At a pH of 6.25, a surprisingly high amount of lactose was removed. Here, after the diafiltration, the ratio of lactose to 6′-SL was 0.30, turning to only 0.16 after the concentration step, a strong removal of the lactose. Even more, if a higher diafiltration factor would be implemented, an even lower amount of lactose could be obtained. Thus, the process of S10 to S22 can - if desired -be used to purify HMOs while reducing the amounts of lactose present due to its role in fermentation.

TABLE 4

Overview of 3 test cell experiments performed with UF permeates with different pHs. All experiments ran a diafiltration with DF = 3 followed by a concentration step with CF = 10

| | pH 4.4 | | pH 5.56 | | pH 6.25 | |
|---|---|---|---|---|---|---|
| | Feed | Retentate | Feed | Retentate | Feed | Retentate |
| Cation analysis | ppm | ppm | ppm | ppm | ppm | ppm |
| NH₄⁺ | 290 | 270 | 270 | 810 | 260 | 990 |
| Ca²⁺ | <3 | <9 | <3 | <9 | <3 | <9 |
| Fe²⁺ | <3 | 9 | <3 | 9 | <3 | 15 |
| K⁺ | 220 | 210 | 200 | 285 | 180 | 525 |
| Mg²⁺ | 6 | 39 | <3 | 9 | <3 | 9 |
| Na⁺ | 170 | 135 | 155 | 180 | 145 | 285 |
| Anion analysis | g/100 g | g/100 g | g/100 g | g/100 g | g/100 g | g/100 g |
| Cl⁻ | <0.001 | <0.003 | <0.001 | <0.003 | <0.001 | <0.003 |
| SO₄²⁻ | 0.001 | <0.003 | <0.001 | 0.003 | <0.001 | 0.003 |
| H₂PO₄⁻ / HPO₄²⁻ | 0.20 | 0.18 | 0.082 | 0.018 | 0.025 | n/a |
| HPLC analysis | g/L | g/L | g/L | g/L | g/L | g/L |
| NANA | 0.15 | 0.87 | 0.05 | 0.79 | 0.08 | 0.87 |
| 6′-SL | 5.2 | 45.16 | 4.4 | 35.69 | 4.94 | 46.66 |
| Lactose | 4.85 | 25.32 | 4 | 15.22 | 5.14 | 7.31 |
| Phosphoric acid | 2.12 | 0.27 | 4.84 | 0.23 | 0.3 | 0.08 |
| Acetic acid | 0.43 | -¹ | 0.67 | -¹ | 0.5 | -¹ |

¹ - not detected

Crossflow nanofiltration experiments using a fermentation broth comprising 6′SL and the solution prepared by steps S10 to S18 thereof using a nanofiltration with a CF of up to 12.6 at a TMP of 28-30 bar and subsequent DF of 2.25, again at a TMP of 28-30 bar were performed.

The results demonstrated that in the nanofiltration of step S22 with a concentration and subsequent a diafiltration step, concentrations of 168 g/l 6′SL were achieved, while ions like chloride, sulphate, monovalent phosphoric acid and phosphate were all below 0.002 wt% in the final retentate. This demonstrates the potential of the method including steps S10 to S22 as an improved process that will allow for purification and concentration of 6′SL and other sialylated HMOS as well as other HMOs without the need for a demineralization step any longer. Lactose can be also retained by this process if desired - in the final retentate the lactose level was around half of the 6′Sl level in g/l.

For Experiments A to D in the following Table B, fermentation broths containing inter alia 2-fucosyl lactose (2-FL) were used. First, the biomass was removed from the broths which were, then, set to a given pH and, in case of Experiment A and D, treated with active carbon (AC). Subsequently, the broths were subjected to concentration using the nanofiltration membrane AMS AS3014 (available from AMS Technologies Ltd., Israel) having a cut-off of 0.4 kDa. In case of Experiment C, the concentrate obtained in Experiment A was diafiltrated using said membrane.

TABLE B

Nanofiltration Experiments

| | | |
|---|---|---|
| Experiment | Feed | Average flux at TMP = 30 bar and 30° C. |
| A | AC-treated broth | 10.3 kg/(m²h) |
| | Set to pH 5.05 using HCl | |
| B | Broth (no AC) | 4.8 kg/(m²h) |
| | Set to pH 5.03 | |
| C | Concentrate of Exp A | 3.7 kg/(m²h) |
| D | AC-treated broth | 5.3 kg/(m²h) |
| | Set to pH 8.88 using NaOH | |

In the feeds used for and the concentrates resulting from nanofiltration, the concentrations of several components were analyzed via HPLC as can be seen in the following Table C:

TABLE C

Analysis Results. Values are concentrations in g/l

| | Exp. A | | Exp. C | Exp. B | | Exp. D | |
|---|---|---|---|---|---|---|---|
| | Feed | Concentrate | After diafiltration concentrate of Exp. A | Feed | Concentrate | Feed | Concentrate |
| 2-Fucosyl lactose (2-FL) | 19.6 | 123.6 | 99.9 | 25.0 | 160.3 | 17.4 | 163.1 |
| Lactose | 12.9 | 87.4 | 64.1 | 12.3 | 84.7 | 12.1 | 84.9 |
| Phosphoric acid | 4.0 | n.a. | 2.3 | 3.5 | n.a. | 3.0 | 13.8 |
| Pyruvic acid | 0.3 | 1.2 | 0.2 | 0.2 | 0.8 | 0.2 | 0.1 |
| Fucose | 0.1 | 0.7 | 0.2 | 0.1 | 0.4 | n.a. | 0.1 |
| Succinic acid | 0.3 | 0.9 | n.a. | 0.5 | 1.1 | 0.4 | 0.8 |
| Lactic acid | 0.3 | 1.5 | 0.5 | 0.6 | 1.2 | 0.4 | 1.4 |
| Formic acid | 0.8 | 0.8 | 0.2 | 1.4 | 1.5 | 0.5 | 0.4 |
| Acetic acid | 2.6 | 1.7 | n.a. | 4.0 | 3.9 | 2.5 | 1.3 |
| Ratio 2-FL / Phosphoric acid | 4.9 | n.a. | 44.4 | 7.1 | n.a. | 5.8 | 11.8 |
| Ratio 2-FL / Formic acid | 25.0 | 162.8 | 463.8 | 18.1 | 108.1 | 33.1 | 465.1 |
| Ratio 2-FL / Acetic acid | 7.6 | 74.5 | n.a. | 6.3 | 41.4 | 6.9 | 122.8 |

n.a. = not available

As can clearly be seen from the increasing ratio of 2-FL to phosphoric, formic or acetic acid, respectively, after concentration or diafiltration, nanofiltration results in a removal of the according deprotonated acids, confirming effective demineralization of the fermentation broths.

### Ion Exchange Experiments

Demineralization experiments in laboratory columns (inner diameter 20 mm) For initial testing of the demineralization procedures, two double-jacketed glass columns (Inner diameter 20 mm, height 1000 mm) were set up and filled with ca. 0.28 L Dowex Monosphere 88 H and 0.24 L Dowex Monosphere 77, respectively.

The demineralization experiments were carried out using the conditions shown in Table 5, with the cation exchange done before the anion exchange. The pre-rinsing, loading, product displacement and post-rinsing steps were carried out with the columns connected in series, first the cation exchanger and then the anion exchanger. The columns and the vessels for the feed and effluent solutions were cooled to ca. 10° C. The regeneration and the rinsing afterwards were carried out in countercurrent mode for each column separately. The resins were regenerated before first use to ensure that they were in a completely regenerated state.

During the experiments, fractions were collected and analysed to monitor the process.

TABLE 5

Conditions for demineralization experiments in the 20 mm laboratory columns

| | | | | |
|---|---|---|---|---|
| Step | Medium | Direction | Amount [BV] | Flow rate [BV/h] |
| Pre-rinsing | Sterile DI water | ▼ | Until conductivity <10 µS/cm | ca. 0.2 (relative to CEX) |
| Loading | Feed | ▼ | See experiment | 0.8 (relative to CEX) |
| Product displacement | DI water | ▼ | 4 (relative to CEX) | 0.8 (relative to CEX) |
| Post-rinsing | DI water | ▼ | 3 (relative to CEX) | ca. 1.6 (relative to CEX) |
| Regeneration | CEX: H2SO4 5 wt% | ▲ | CIEX: 8 | 3 |
| | AEX: NaOH 4 wt% | | AIEX: 10 | |
| Rinsing | DI water | ▲ | ca. 20 | ca. 1.6 |

### Demineralization of LNT Samples

Dowex Monosphere 88 H and Dowex Monosphere 77 were chosen as these have been used for oligosaccharides before. Their properties are shown in Table 6. The supplier has recently renamed these products and they are now being sold as AmberLite FPC88 UPS H and AmberLite FPA77 UPS, respectively.

TABLE 6

Properties of the ion exchangers used for demineralization

| | | |
|---|---|---|
| | Cation exchanger | Anion exchanger |
| Supplier | Dupont | Dupont |
| Name | Dowex Monosphere 88 H (AmberLite FPC88 UPS H) | Dowex Monosphere 77 (AmberLite FPA77 UPS) |
| Type | Strong acid cation | Weak base anion |
| Matrix | Styrene-DVB, macroporous | Styrene-DVB, macroporous |
| Functional group | Sulfonate | Tertiary amine with some quarternary groups |
| Delivery form | H | free base |
| Total exchange capacity | min 1.7 eq/L | min 1.7 eq/L, |
| | | min 1.5 eq/L as weak base |
| Water content | 50-56 % | 40-50% |
| Particle size distribution | Median 500-600 µm with 95% within 400-720 µm | Median diameter 475-600 µm |
| Swelling Na->H | 5% | |
| Swelling FB->HCl | | 22% |
| Whole beads | min 95% | min 95% |
| Particle density | 1.2 | 1.04 |
| Shipping weight | 770 g/L | 640 g/L |

Samples of UF permeate (i.e. permeates of the second membrane filtration step S18) and of NF retentates obtained from treating the UF permeate with two different methods of nanofiltration were (step S22) were analysed.

The final retentates from the experiments 002 and 003 (see above) where used for ion exchange experiments.

The results of the demineralization experiments are summarized in Table 7. As can be seen in the table, the amount of salt relative to the product was reduced considerably in nanofiltration, and with additional washing it could be reduced even further.

TABLE 7

Properties of the UF permeate and NF retentate with and without additional washing

| | Control sample (UF permeate) | | experiment 002 (NF retentate) | | experiment 003 (NF retentate, washed) | |
|---|---|---|---|---|---|---|
| Conductivity (mS/cm) | | 6.8 | | 8.1 | | 3.9 |
| APHA | | 430 | | 2760 | | 2630 |
| pH | | 3.7 | | 3.8 | | 4.0 |
| | g/L | | g/L | Factor | g/L | Factor |
| Lacto-N-tetraose | | 12.88 | 106.52 | 8.27 | 120.73 | 9.37 |
| Lacto-N-triose | | 0.44 | 3.36 | 7.62 | 3.50 | 7.95 |
| Lactose | | 0.96 | 5.25 | 5.46 | 3.79 | 3.94 |
| | ppm | eq/kg | ppm | eq/kg | ppm | eq/kg |
| NH₄⁺ | 140 | 0.008 | 130 | 0.007 | 10 | 0.001 |
| Ca²⁺ | 7 | 0.000 | 27 | 0.001 | 14 | 0.001 |
| Fe²⁺ | 4 | 0.000 | 14 | 0.001 | 10 | 0.000 |
| K⁺ | 1300 | 0.033 | 1300 | 0.033 | 75 | 0.002 |
| Mg²⁺ | 34 | 0.003 | 155 | 0.013 | 105 | 0.009 |
| Na⁺ | 290 | 0.013 | 310 | 0.013 | 20 | 0.001 |
| SUM Cations | 1775 | 0.057 | 1936 | 0.069 | 234 | 0.013 |

| | | | | | | |
|---|---|---|---|---|---|---|
| | % | eq/kg | % | eq/kg | % | eq/kg |
| Cl⁻ | <0.001 | 0.000 | 0.001 | 0.000 | <0.001 | 0.000 |
| SO₄²⁻ | 0.26 | 0.054 | 0.62 | 0.129 | 0.33 | 0069 |
| H₂PO₄⁻ | 0.15 | 0.016 | 0.21 | 0.022 | 0.05 | 0.005 |
| SUM Anions | 0.41 | 0.070 | 0.83 | 0.151 | 0.38 | 0.074 |
| g salt/g LNT | | 0.46 | | 0.10 | | 0.03 |
| Cation/anion charge ratio | | 0.81 | | 0.45 | | 0.18 |

A control sample (no nanofiltration treatment after the ultrafiltration as second membrane filtration) was used as feed. The feed was loaded onto the ion exchange columns at a flow rate of 0.8 BV/h relative to the cation exchanger until a conductivity of 50 µS/cm was reached in the effluent. This took place after approximately 18 BV, however a breakthrough in colour was observed already after 16 BV. The conductivity of the effluent throughout the process was approximately 15 µS/cm indicating a small leakage of ions. Accordingly, the pH of the effluent was slightly alkaline since the salt leakage causes a small amount of hydroxide ions to be displaced from the anion exchanger. The reason for this leakage is not clear but may be that other components in the mixture can form complexes with some of the ions. The anion exchanger was observed to swell by approximately 5% during the loading and the cation exchange to shrink by a few percent.

The washed NF retentate (from experiment 003 above) was demineralized at a flow rate of 0.8 BV/h using the same columns as for the control samples after their regeneration. In this experiment, a predetermined amount of feed of the solution from experiment 003 was passed through the columns, 10 BV.

The elution of LNT was completed earlier than for the control sample.

As for the control sample, the breakthrough in colour came somewhat earlier, after 6.2 BV, and like previously with the control sample, also here a small leakage of ions was observed during the run. Also, in this case the anion exchanger was observed to swell by approximately 5% during the loading and the cation exchange to shrink by a few percent.

The colourless fractions of each demineralization experiment were combined per experiment. The combined fractions of the control sample and the combined fractions of the nanofiltrated sample from experiment 003 were then analysed. The results (see Table 8) showed that the pre-treatment with nanofiltration and washing resulted in lower residual levels of ions relative to the fine chemical LNT.

TABLE 8

Analysis of demineralized products

| | Colourless Fractions control sample | Colourless Fractions washed NF retentate exp 003 |
|---|---|---|
| Conductivity | 16 µS/cm | 15 µS/cm |
| APHA | 0.4 | 1.1 |
| pH | 8.2 | 8.1 |
| Lacto-N-tetraose | 12.75 g/L | 92.22 g/L |
| Lacto-N-triose | 0.35 g/L | 2.50 g/L |
| Lactose | 0.85 g/L | 2.66 g/L |
| NH₄⁺ | <10 ppm | <10 ppm |
| Ca²⁺ | <1 ppm | <1 ppm |
| Fe²⁺ | <1 ppm | <1 ppm |
| K⁺ | 5 ppm | 6 ppm |
| Mg²⁺ | <1 ppm | <1 ppm |
| Na⁺ | 1 ppm | <1 ppm |
| Chloride | <0.001% | <0.001% |
| Sulfate | <0.001% | <0.001% |
| Phosphate | <0.001% | <0.001% |

It was observed that the throughput was much higher when the NF retentate with reduced amounts of salts was used in the demineralization step, 167% more of LNT per cycle with a cycle time that was reduced by almost 60%, i.e. a total improvement by about 350%. Thus, when the step of nanofiltration including the washing of the samples was used, the throughput of the desired fine chemical in the demineralization step was improved by a factor of about 4.5 compared to the throughput of the demineralisation of the control sample that had not undergone any nanofiltration after the ultrafiltration as second membrane filtration. As shown in the experiments, the efficiency of the demineralization was also not compromised, and less residual ions relative to the fine chemical LNT were obtained when the washed NF retentate was demineralized compared to the control sample without NF treatment.

It was observed that the concentration step alone (experiment 002) does not change the ion concentrations at large; however, since the LNT concentration was increased by a factor 8.3, the relative concentration of ions to LNT was reduced significantly (see Table 7). For experiment 003, the extra diafiltration step results in a strong decrease in ion concentration, especially in the concentration of the monovalent K⁺ and H₂PO₄⁻. The divalent SO₄²⁻ ion is reduced to a lesser extent and the divalent Mg²⁺ is only reduced to a small extent.

As demonstrated nanofiltration before ion exchange can be used to remove nearly all the phosphoric acid and parts of the lactose from the solution or remove ions preferably but not the lactose or LNT. Furthermore, the broth can be concentrated by at least a factor 10, probably more, judging from the flow rates at the end of the concentration step. The currently employed concentration factors of 10 followed by a diafiltration factor of 3 allow for a removal of -65% of the lactose and >95% of the phosphoric acid. Using higher diafiltration factors, higher removal rates of lactose are achievable for the person skilled in the art.

### Summary of the Results of the Demineralization Experiments

Carrying out NF before demineralization has been found to have several advantages:
Considerably higher throughput during ion exchange, up to 350% higher
If desired, lactose could be partially removed during the nanofiltration which demonstrated also improved purification of the product LNT
Less residual salt after demineralization relative to the product were found when nanofiltration was employed

Overall, the inventive method to combine decolourization, biomass removal, purification by nanofiltration and ion exchange proved to be very efficient on resources and equipment while delivering fast recovery of a number of fine chemical products such as different HMO types.

### CITED LITERATURE

WO 2015/032412
EP 2 379 708
CN 100 549 019 & CN 101 003 823
WO 2017/205705
EP 2 896 628
US 9 944 965
WO 2019/003133
WO2015106943

1-17. (canceled)

18. A method for the demineralization of a solution comprising one or more fine chemicals, wherein the method comprises the steps of:
a. providing the solution comprising one or more oligosaccharides;
b. optionally adjusting the pH to the desired value below 7 or less by adding at least one acid to the solution comprising at least one oligosaccharide,
c. optional decolourisation step,
d. an optional incubation step,
e. carrying out a first membrane filtration,
f. a second membrane filtration of the permeate of the first membrane filtration,
g. optional decolourisation step of the permeate of the second membrane filtration,
h. a first nanofiltration step; with a sub-step of concentration and/or a sub-step of diafiltration.

19. The method according to claim 18, wherein steps b) and c) are performed.

20. The method according to claim 18, wherein no cation exchange or anion ion exchange is carried out.

21. The method according to claim 18, wherein in step h), a sub-step of concentration and a sub-step of diafiltration is carried out.

22. The method according to claim 18, wherein the concentration sub-step of step h) is performed so that the concentration factor is at least 3 or more.

23. The method according to claim 18, wherein the diafiltration sub-step of step h) is performed so that the diafiltration factor is from 2.5 to 3.5.

24. The method according to claim 18, wherein the nanofiltration membrane has a NaCl retention between 5 to 30%.

25. A method for purification of one or more fine chemicals from a solution comprising biomass and one or more oligosaccharides, comprising the steps of
i. providing a solution comprising biomass and one or more oligosaccharides,
ii. setting the pH value of the solution below 7 or less by adding at least one acid to the solution comprising biomass and the at least one oligosaccharide,
iii. decolourizing the solution at least in part by adding an adsorbing agent to the solution comprising biomass and one or more oligosaccharides,
iv. optionally an incubation step,
v. carrying out a membrane filtration also called herein the first membrane filtration and typically being a microfiltration or ultrafiltration so as to separate the biomass from the solution comprising the one or more oligosaccharides;
vi. optionally carrying out at least one second or further membrane filtration with the permeate of the first membrane filtration;
vii. optionally carrying out a decolourization step;
viii. carrying out a nanofiltration with the permeate of the membrane filtration antedating this step viii, either with the permeate of the first or the second or any further membrane filtration;
ix. optionally a decolourization step;
x. optionally a second nanofiltration with the retentate of the nanofiltration of the previous nanofiltration step viii, wherein the nanofiltration membrane used is a different one to the one in the previous nanofiltration step;
xi. optionally a third or further nanofiltration with a membrane differing from the one of the previous nanofiltration step.

26. The method of claim 25, with subsequent to step xi further processing of the retentate of the previous nanofiltration step by any of the following steps is conducted:
a. carrying out a decolourization step, and / or
b. carrying out a demineralization step, and / or
c. carrying out an electrodialysis and / or reverse osmosis and / or concentration step and / or a decolourization step; and / or
d. carrying out a simulated moving bed chromatography, and / or a solidification step creating a solid oligosaccharide product, and / or a spray drying of the oligosaccharide followed by drying as desired.

27. The method according to claim 26 wherein a demineralization step is conducted after the last one of the one or more nanofiltrations of steps viii, x and xi, wherein the demineralization is performed by ion exchange and wherein further the throughput of the demineralisation step is increased by a factor of at least 2 or more compared to the throughput of an identical ion exchange step without the one or more nanofiltrations of steps viii, x and xi preceding it.

28. The method according to claim 18, wherein the pH value of the solution is lowered to a pH value in the range of 3.0 to 5.5.

29. The method according to claim 18, wherein said adsorbing agent is added in an amount in the range of 0.5% to 3% by weight.

30. The method according to claim 18, wherein said first membrane filtration is carried out as cross-flow microfiltration or cross-flow ultrafiltration.

31. The method according to claim 18, wherein said first membrane filtration is carried out at a temperature of the solution in the range of 8° C. to 55° C.

32. The method according to claim 18, further comprising carrying out a second membrane filtration with the solution comprising oligosaccharides obtained by the first membrane filtration.

33. The method according to claim 31, wherein said second membrane filtration is carried out at a temperature of the solution being in the range of 5° C. to 15° C.

34. The method according to claim 18, wherein said at least one oligosaccharide comprises human milk oligosaccharide.
